(19) United States
(12) Patent Application Publication
(10) Pub. No.: US 2026/0117167 A1
(43) Pub. Date: Apr. 30, 2026

(54) HYBRID MUSHROOM STRAIN B19414 AND METHODS AND USES THEREFOR

(71) Applicants: Sylvan Inc., Kittanning, PA (US); Sylvan America, Inc., Kittanning, PA (US)

(72) Inventors: Michelle Schultz, New Bethlehem, PA (US); Michael Kessler, Kittanning, PA (US); Mark Loftus, Oakmont, PA (US); Aniça Amini, Saint Pierre Des Corps (FR); Sylvie Delbecque, Langeais (FR); Wes Schultz, Worthington, PA (US); Mark Wach, Allison Park, PA (US)

(21) Appl. No.: 18/848,264

(22) PCT Filed: Mar. 23, 2023
(86) PCT No.: PCT/US2023/016014

§ 371 Date: Sep. 18, 2024

Related U.S. Application Data
(60) Provisional application No. 63/322,793, filed on Mar. 23, 2022.

(51) Int. Cl. C12N 1/14; C12R 1/645

(52) U.S. Cl. CPC C12N 1/145; C12R 2001/645

(57) ABSTRACT

A hybrid mushroom culture of Agaricus bisporus, designated as strain B19414, includes a representative culture of the strain, which has been deposited under NRRL Accession No. 68095. A method of producing a hybrid mushroom culture of Agaricus bisporus comprising: mating a homokaryotic line designated B12998-s181, a culture of which has been deposited under NRRL Accession No. 68094, with a homokaryotic line designated P2-s203, a culture of which has been deposited under NRRL Accession No. 68093. Additionally, Essentially Derived Varieties, mushrooms, parts of the culture, products incorporating the culture and uses of the cultures are provided.

### CROSS REFERENCE TO RELATED APPLICATIONS

This application claims the benefit of U.S. Provisional Patent Application No. 63/322,793, filed Mar. 23, 2022, the disclosure of which is incorporated herein by reference.

### FIELD OF THE INVENTION

This invention relates to a novel class of cultures of the edible, cultivated mushroom fungus Agaricus bisporus (Lange) Imbach, and methods of producing and using said cultures. More particularly, this invention relates to a newly developed hybrid strain designated B19414 and to cultures that are descended or, separately, derived from Agaricus bisporus strain B19414, including Essentially Derived Varieties.

### BACKGROUND OF THE INVENTION

The button mushroom Agaricus bisporus (Lange) Imbach var. bisporus, a microorganism belonging to the basidiomycete fungi, is widely cultivated around the world as a food item. In Europe and North America, it is the most widely cultivated mushroom species. Agaricus bisporus cultivars are dominated by two mushroom cap colors; white and brown. Furthermore, the market is segmented into two marketing categories; medium-sized (25 to 50 mm) closed caps, or buttons (in browns, these are often called creminis) and open or flat mushrooms, which are the mature stage of mushroom fructification. ‘Breakfast flats’ and ‘portobellos’ are two established marketing categories for open or flat mushrooms.

Cultures of Agaricus, like those of other microorganisms, are prepared, maintained, propagated, and stored on sterile media using microbiological laboratory methods. Sterile tools and aseptic techniques are used within clean rooms or sterile transfer hoods to manipulate cells of the pure cultures for various purposes including clonal propagation and for the development of new strains using diverse techniques including spore germinations on sterile growth media and controlled matings on sterile growth media. Commercial culture inocula including mushroom ‘spawn’ and ‘casing inoculum’ are also prepared using large-scale microbiological production methods, for example by aseptically introducing inoculum of a pure culture of a strain of Agaricus bisporus into from one to 14,000 liters of sterilized growth media under sterile conditions and are provided to the end user as pure cultures on sterile growth media contained within sterile packaging.

Mushrooms are cultivated commercially within purpose-built structures on dedicated farms. While there are many variations on methods, the following description is typical. Compost prepared from lignocellulosic material such as straw, augmented with nitrogenous material, is finished, and pasteurized within a suitable facility. Mushroom spawn, which comprises a sterilized friable ‘carrier substrate’ onto which a pure culture of one mushroom strain has been aseptically incorporated via inoculum and then propagated, is mixed with the pasteurized compost, and is incubated for approximately 13 to about 19 days at a controlled temperature, during which time the mycelium of the mushroom culture colonizes the entire mass of compost and begins to digest it. A non-nutritive ‘casing layer’ of material such as peat mixed with limestone is then placed over the compost to a depth of from about 40 to 50 mm. Additional ‘casing inoculum’ incorporating the same mushroom culture may be incorporated into the casing layer to accelerate the formation and harvesting of mushrooms, and also to improve uniformity of the distribution of mycelium and mushrooms in and on the casing surface. Environmental conditions, including temperature and humidity in the cropping facility are then carefully managed to promote and control the transition of the culture from vegetative to reproductive growth at the casing/air interface. In a further 13 to 18 days after casing, mushrooms will have developed to the correct stage for harvest and sale. This is called a flush or break. A first flush or first break of mushrooms comprising the original culture may be picked over a 3 to 4 day period. Additional flushes of mushrooms (i.e., second-flushes or third-flushes, or second-breaks or third-breaks) appear at about weekly intervals. Commercially, two or three flushes or breaks of mushrooms are produced and harvested before the compost is removed and replaced in the cropping facility.

Generally speaking, strains may be differentiated on the basis of traits associated with the mushroom, such as mushroom size (including cap diameter), mushroom shape (e.g., cap roundness, flesh thickness), color (i.e., white cap versus brown cap), surface texture (e.g., cap smoothness), tissue density and/or firmness, delayed maturation, basidial spore number greater than two, sporelessness, increased dry matter content, improved shelf life, and reduced bruising, as well as traits associated with the culture itself, and/or products incorporating the culture, and/or crops incorporating the culture, including increased crop yield, altered distribution of yield over time, decreased spawn to pick interval, resistance to infection by, symptoms of, or transmission of bacterial, viral or fungal diseases, insect resistance, nematode resistance, ease of crop management, suitability of crop for mechanical harvesting, and behavioral responses to environmental conditions including stressors, nutrient substrate composition, seasonal influences, farm practices, self/non-self interactions (compatibility or incompatibility) with various mushroom strains, to give some examples. Strains may also be differentiated based on their genotypic fingerprint (presence of specific alleles at defined marker loci in the nuclear or mitochondrial genome). Strains may have different ancestry, which will be reflected directly by the genotype, and indirectly, in some cases, by the phenotype.

Five to thirty percent of the Agaricus mushrooms cultivated in the United States. Europe, and elsewhere have a brown pileus color, in accordance with consumer preferences for a traditional or ‘old-fashioned’ product appearance. The ‘portobello’ mushroom market segment is supplied by the cultivation of brown-capped (=brown) strains. Market requirements for brown mushrooms in the USA and elsewhere are relatively narrow and precise for many observable phenotypic traits such as size, shape, color, color retention, firmness, and related traits such as shelf life. Consequently, genetically different strains of commercially successful brown Agaricus bisporus mushrooms that may be differentiated on the basis of appearance of the mushrooms, can, in some instances, be important.

Circa 1980, the first two white hybrid strains of A. bisporus, developed by a laboratory at Horst, the Netherlands, were introduced into commercial cultivation. These two “Horst” strains, called U1 and U3, are closely related hybrid strains produced by matings between two pre-existing white cultivated strains, as per M. Imbernon et al., Mycologia, 88 (5), 749-761 (1996), herein incorporated by reference. The two parents of U1 and U3 are commercial strains belonging to two longstanding categorical types of strains known as the ‘smooth-white’ (SW) strains and the ‘off-white’ (OW) strains. The original homokaryons (or ‘lines’) obtained from the SW and OW strains, and used in the hybridization that produced the U1 strain, were designated H39 and H97 respectively; these cultures may no longer exist (A. Sonnenberg, pers. comm.).

However, a number of laboratories have deheterokaryotized the U1 strain and obtained neohaplont cultures incorporating one or the other nuclear type corresponding to those contributed by H39 or H97, as well as the mitochondrial type of U1. The industry refers to these two types of neohaplonts of U1 categorically as the SWNC and OWNC lines or homokaryons, respectively. An OWNC line designated ‘H97’ was deposited in the public culture collection of the Fungal Genetics Stock Center of Kansas, USA, by A. Sonnenberg, under the number 10389, and in the public collection of the American Type Culture Collection of Maryland, USA, under the number MYA-4626. The genome of H97 was sequenced and placed in the public domain by the Joint Genome Institute of California, USA. (See Morin et al. 2012, herein incorporated by reference).

One traditional type of brown-capped strain of A. bisporus mushroom, most often called the ‘Old-Fashioned Brown’ strain (or ‘OFB’; examples of the OFB strain type include Sylvan's SB-65, SB-295, and RWK-2042 strains), originated as a wild strain in Europe and was the leading brown cultivar strain for many decades, even becoming the only brown cultivar in wide use in the last years of the twentieth century. A few different brown-capped hybrid strains have been developed since the 1980s, and some have enjoyed some commercial success.

Heterokaryotic spores of an initial strain retain the great majority of the parental genotype (this behavior was shown by R. W. Kerrigan et al. in Genetics, 133, 225-236 (1993), herein incorporated by reference). A group of strains developed either by cloning or by spore culture, or by any other method of ‘essential derivation’ as discussed below, from a single progenitor (as opposed to outbreeding between two different progenitors) is called a derived lineage group. Many commercial mushroom strains developed from the OFB stock meet the criteria for Essentially Derived Varieties (as the term is applied to plant varieties).

Agaricus bisporus has a reproductive syndrome known as amphithallism, in which two distinct life cycles operate concurrently. As in other fungi, the reproductive propagule is a spore. Agaricus produces spores meiotically, on a meiosporangium known as a basidium. In a first life cycle, A. bisporus spores each receive a single haploid post-meiotic nucleus; these spores are competent to mate but not competent to produce mushrooms. These haploid spores germinate to produce homokaryotic offspring or lines which can mate with other compatible homokaryons to produce novel hybrid heterokaryons that are competent to produce mushrooms. Heterokaryons generally exhibit much less ability to mate than do homokaryons. This life cycle is called heteromixis, analogous to outbreeding. This life cycle operates but typically does not predominate in strains of Agaricus bisporus var. bisporus.

A second, life cycle called intramixis, analogous to a form of inbreeding, predominates in most strains of Agaricus bisporus var. bisporus. Most spores receive two post-meiotic nuclei, and most such pairs of nuclei consist of Non-Sister Nuclear Pairs (NSNPs) which have a heteroallelic genotype at most or all centromeric-linked loci including the MAT locus. That MAT genotype determines the heterokaryotic phenotype of these offspring, which are reproductively competent and can produce a crop of mushrooms. Unusually among eukaryotes, relatively little (if any) chromosomal crossing-over is observed to have occurred in postmeiotic offspring of A. bisporus: empirically, very little heteroallelism (analogous to heterozygosity) is lost among heterokaryotic offspring of a heterokaryotic strain.

Functionally, low rates of recombination occur over much of each chromosome, in conjunction with intramixis, leading to parental and intramictic offspring heterokaryotic genotypes and phenotypes that tend to closely resemble each other. This provides a preferred method for deriving, from an initial culture, novel cultures having trivial, minor genetic alterations while retaining the important characteristics of the initial culture. Such cultures derived by this or alternate methods, solely or predominantly (as in the case of back-mating) from one initial culture, are called Essentially Derived Varieties (also known by the initialism, EDVs). EDVs may be virtually indistinguishable from that initial culture. Derivation is a familiar strain development technique in the art, among commercial mushroom spawn producers, particularly when the commercial objective is to obtain and exploit a ‘knockoff’ of a protected culture without simply cloning or copying the original culture.

As noted above, the first hybrid strains of A. bisporus, developed by a laboratory at Horst, the Netherlands, via the heteromictic life cycle, were introduced circa 1980 into commercial cultivation. These two white “Horst” strains, called U1 and U3, were derived from matings between two pre-existing white cultivated strains, and following the successful commercial release of the white U1 strain and associated Essentially Derived Varieties, (defined herein), the first commercially successful hybrid brown cultivar entered the marketplace in the early 1990s with the release of Sylvan 600, patented as X618 (U.S. Pat. No. Plant 7,636). Beginning around 2008 the most widely marketed commercial brown cultivar has been Heirloom, a strain protected by U.S. Pat. No. 7,608,760. In 2014 Sylvan's Tuscan Brown cultivar strain (also known as Tuscan-860 and as B14528 in U.S. Pat. No. 9,642,333) was also successfully released.

Also, as noted above, the market for mushrooms is segmented into two broad categories: closed caps and flat mushrooms. Current brown strains such as Heirloom. Tuscan-820 and Tuscan-860 are adequate flat mushroom performers, but the need exists for strains that have thinner cap dimensions and larger cap diameters, which tend to make better portobellos or breakfast flats. In other words, wider and flatter mushroom shape and thinner cap dimensions are desirable in two key mushroom sales niches: the North American portobello market, and the UK/Ireland flat mushroom market. Flat mushrooms are handled in the home and restaurant kitchens very differently to buttons. For example, in North America portobellos are typically marinated and grilled on a barbeque and are generally consumed as an alternative to meat. In the UK and Ireland, flat, open mushrooms are fried and form part of the traditional Irish or British “fried breakfast”. For both of these applications, a flatter shape is preferred. For ease of discussion, henceforth, a reference to “portobellos” or “portobello mushrooms” will mean any brown-capped, flat mushrooms as distinguished from closed caps, or button mushrooms.

In the UK and Ireland, it is believed that 26-30% of the first break mushrooms are harvested as either “breakfast flats” or portobellos, with a smaller amount of second break product also entering the flat market. In fact, 4.2 million pounds of portobellos are grown annually in UK and Ireland and with farm gate value of 4.2 million Euros. These mushrooms may be brown or white.

The US and Canadian markets tend to use brown strains to grow larger sized (70 to 100 mm), portobellos. In 2019-2020, US brown mushroom production was 183.7 million pounds (USDA NASS). Approximately 35-40% of this total production was portobellos (between 64 to 73.5 million pounds). These larger mushrooms brought in a gross value of between $99-104 million.

Smaller mushrooms, and later break mushrooms, from such crops are marketed as buttons. For example, about two-thirds of the total brown crop is harvested as closed button mushrooms. From the foregoing information it will be appreciated that any mushroom strain used to produce large open or flat mushrooms must also be capable of producing good quality button mushrooms in sufficient quantity to provide a product mix that is optimally profitable.

Hygiene is very important in protected (i.e. enclosed) crops such as mushrooms. Mushroom viruses, in particular La France disease and Mushroom Virus X, can be devastating diseases and are spread via infected spores or contaminated compost substrate. If a grower switches to a strain which is vegetatively incompatible with the previously used strain, then the two major routes of spread can be blocked by exploiting the dramatic reduction of anastomoses (cell fusions) between incompatible heterokaryotic strains. The end result will be a disease ‘breaking,’ and a recovery in yield and quality. Thus, one of the advantages of having available for use more than one strain is the option of using an alternate strain to achieve a disease-break for highly transmissible diseases such as mushroom virus. More generally, it is well established that crop monocultures create a large risk of widespread crop failures due to having common vulnerabilities. Generic diversification of crops in production manages this monocultural risk. Having multiple, genetically diverse, commercially acceptable options provides a solution to this problem.

Furthermore, there is a need for a strain of Agaricus bisporus which produces a crop of mushrooms which have brown caps and have a three break yield at least equivalent to that of an accepted commercial strain, specifically the Heirloom strain, a representative culture of the Heirloom strain having been deposited in the American Type Culture Collection (ATCC) under the name BR06 under ATCC Accession No PTA-6876 and are disclosed in U.S. Pat. No. 7,608,760. The need further exists to produce Agaricus bisporus strains that produce mushrooms having wider cap diameters than other well-known commercial brown-capped mushrooms, including Heirloom (see above), Tuscan-860, a representative culture of the Tuscan-860 strain having been deposited in the Agriculture Research Service Culture Collection (NRRL) under NRRL Accession No. 50900, Tuscan-820, a representative culture of the Tuscan-820 strain having been deposited in the Collection Nationale de Cultures de Microorganismes (CNCM), Paris, France, under CNCM Accession No. 1-5527, and Brawn, a representative culture of the Brawn strain having been deposited in the Agriculture Research Service Culture Collection (NRRL) under NRRL Accession No. 68258.

Still further, there is a need to produce Agaricus bisporus strains that produce mushrooms having thinner Flesh Thickness and Cap Flatness at maturity, or that have a smaller proportion of stem tissue, compared to Heirloom. There is a further need for a strain having the foregoing attributes while also having genetic novelty to enhance crop diversification.

### SUMMARY OF THE INVENTION

The present invention is generally directed to a new and distinct Agaricus bisporus mushroom culture designated B19414; a hybrid strain obtained via the directed mating of two homokaryotic cultures. A deposit of a culture of strain B19414 has been made with the Agricultural Research Services Culture Collection (NRRL), 1815 North University Street, Peoria, Illinois 61604 USA. The date of deposit was Mar. 9, 2022. The culture deposited was taken from the same culture maintained by Sylvan Inc., Kittanning. Pennsylvania, USA, the assignee of record, since prior to the filing date of this application. All restrictions upon the deposit have been removed, and the deposit is intended to meet all deposit requirements of the U.S. Patent and Trademark Office, including 37 C.F.R. Sec. 1.801-1.809, and all deposit requirements under the Budapest Treaty. The NRRL Accession Number is 68095. The deposit will be maintained in the depository for a period of 30 years, or 5 years after the last request, or for the effective life of the patent, whichever is longer, and will be replaced as necessary during that period. The cultures have been irrevocably and without restriction or condition released to the public upon the filing the priority application or upon the issuance of a patent on this strain according to the patent laws.

Strain B19414 was obtained via direct mating of two homokaryon parents, namely, line B12998-s181 and line P2-s203. A deposit of a culture of line B12998-s181 has been made with the Agricultural Research Services Culture Collection (NRRL), 1815 North University Street, Peoria, Illinois 61604 USA. The date of deposit was Mar. 9, 2022. The culture deposited was taken from the same culture maintained by Sylvan Inc., Kittanning, Pennsylvania, USA, the assignee of record, since prior to the filing date of this application. All restrictions upon the deposit have been removed, and the deposit is intended to meet all deposit requirements of the U.S. Patent and Trademark Office, including 37 C.F.R. Sec. 1.801-1.809, and all deposit requirements under the Budapest Treaty. The NRRL Accession Number is 68093. The deposit will be maintained in the depository for a period of 30 years, or 5 years after the last request, or for the effective life of the patent, whichever is longer, and will be replaced as necessary during that period. The cultures have been irrevocably and without restriction or condition released to the public upon the filing the priority application or upon the issuance of a patent on this strain according to the patent laws.

Likewise, a deposit of a culture of line P2-s203 has been made with the Agricultural Research Services Culture Collection (NRRL), 1815 North University Street, Peoria, Illinois 61604 USA. The date of deposit was Mar. 9, 2022. The culture deposited was taken from the same culture maintained by Sylvan Inc., Kittanning, Pennsylvania, USA, the assignee of record, since prior to the filing date of this application. All restrictions upon the deposit have been removed, and the deposit is intended to meet all deposit requirements of the U.S. Patent and Trademark Office, including 37 C.F.R. Sec. 1.801-1.809, and all deposit requirements under the Budapest Treaty. The NRRL Accession Number is 68094. The deposit will be maintained in the depository for a period of 30 years, or 5 years after the last request, or for the effective life of the patent, whichever is longer, and will be replaced as necessary during that period. The cultures have been irrevocably and without restriction or condition released to the public upon the filing the priority application or upon the issuance of a patent on this strain according to the patent laws.

Accordingly, the present invention includes a method of producing a mushroom culture of Agaricus bisporus comprising mating a homokaryotic line designated B12998-s181, a culture of which has been deposited under NRRL Accession No. 68094, with a homokaryotic line designated P2-s203, a culture of which has been deposited under NRRL Accession No. 68093.

Cultures of strain B19414 are noted to include mushrooms, parts of mushrooms, including spores, and parts of the culture, including caps, stems, gills, cells, nuclei, mitochondria, protoplasts, cell walls and cell membranes, hyphae, and mycelium. Cultures of strain B19414 are noted to produce, or be able to produce, strains and lines, which are thereby derived from at least some of such cultures. Thus, the present invention encompasses cultures of strain B19414, namely, mushrooms and parts of mushrooms, including spores, produced by strain B19414, Essentially Derived Varieties (EDVs) (defined below, and generally including cultures derived solely or predominantly [as in repeated back-mating] from an initial culture of strain B19414), dormant or active growing cultures present in dormant or germinating spores of strain B19414, and cultures incorporating the genetic material of strain B19414. Cultures of strain B19414 are noted to produce, or be able to produce, crops of edible mushrooms with characteristics noted below. The present invention is also directed towards methods of making and using strain B19414. Uses of cultures of B19414 and other cultures noted above include their incorporation into commercial products such as mushroom spawn and casing inoculum, as well as for the production of mushrooms, the development of additional novel cultures of A. bisporus, and for crop diversification and farm hygiene including ‘virus breaking.’

With respect to spores, living spores are heterokaryons or homokaryons in a dormant state. Spores are one part of the mushroom organism culture; they incorporate only the genetic material of the single culture that produces them (often called the ‘parent’ for convenience). Other parts of the culture include caps, stems, gills, cells (defined as hyphal compartments incorporating nuclei, mitochondria, protoplasts, cell membranes, and cell walls including crosswalls), hyphae, and mycelium. Spores may be aseptically collected on sterile material, suspended in sterile water at various dilutions, and plated onto sterile agar growth media in order to produce germinated spores and the cultures incorporated within the spores. A preferred technique is to have within the enclosed petri plate a living Agaricus culture which may stimulate spore germination via the diffusion of a volatile pheromone. Germinated spores may be isolated under a microscope using sterile microtools such as steel needles, onto fresh nutrient agar plates. Using this method, heterokaryotic and homokaryotic offspring of strain B19414 comprising the spores and the cultures incorporated within the spores of strain B19414 may be obtained.

Development of novel hybrid varieties via heteromixis comprises the controlled physical association and mating of two compatible cultures to obtain a novel heterokaryon culture. Homokaryons (=‘lines’) are the preferred starting cultures for making matings as they have maximal ability to anastomose and achieve plasmogamy with other cultures. Heterokaryons may also be placed in physical contact but with commercially unreasonably low probabilities of a mating resulting in successful formation of a novel heterokaryon. Compatibility is determined by the genotype at the MAT locus; two homokaryons with the same MAT allele cannot establish a heterokaryon after anastomosis, thus homokaryon compatibility represents genetic dissimilarity. In a defined mating program, homokaryotic lines are obtained and are associated in predetermined pairwise combinations. In one method, homokaryon pairs may be placed in close proximity on the surface of a nutrient agar medium in a petri dish and allowed to grow together (in a physical association), at which point anastomoses between the two cultures occur. A successful outcome is a mating that provides a heterokaryon. The novel hybrid heterokaryon may be obtained by transferring mycelium from the fusion zone of the dish. Such a paired mating method, between B12998-s181 and P2-s203 was used to obtain the strain B19414.

Advantageously, strain B19414, due to thinner cap flesh, tends to make better portobellos or breakfast flats. Strain B19414 produces mushrooms with a thinner cap flesh, resulting in a flatter shape. Furthermore, strain B19414 is advantageously a mushroom strain that is used to produce large open or flat mushrooms, but is also capable of producing good quality button mushrooms in sufficient quantity to provide a product mix that is optimally profitable.

Further, strain B19414 has been advantageously found to produce portobello mushrooms at a first break that have a wider diameter than any other commercial brown mushroom, including those selected from the group consisting of Heirloom, Tuscan-820, Tuscan-860 and Brawn, at the same stage of maturity. It will be appreciated that cap diameter is measured at the time that the veil of the mushroom ruptures.

Mushroom morphology can be divided into four broad categories; the cap, the gills, the stem and the veil. For all Agaricus mushrooms, both browns and whites, the goal is to harvest mushrooms before the veil breaks. There are two reasons for this. First, mushrooms with open veils will lose moisture and weigh less, thereby affecting harvested weight and ultimately profits. Second, mushrooms with broken veils will start to shed spores, and this can inhibit the growth of new mushrooms. More importantly, shedded spores are known to spread several mushroom viruses which can have devastating effects on yield and quality.

Additionally, if the mushrooms are left on the beds past the point of veil rupture, the cap margin will begin to curl upwards, as spores are released. Growers favor harvesting when the cap margin underside is “rolled under”, and this occurs when the veil is partly or completely attached.

Still further, advantages are found in strain B19414 that produce mushrooms that have a Flesh Thickness ratio less than 0.49, and in some embodiments, less than 0.40.

Strain B19414 may also produce homokaryons and heterokaryons, and thus, homokaryons and heterokaryons may be obtained from the strain and its EDVs as defined below. Some homokaryons have been obtained from Strain B19414, including homokaryons B19414-s9 and B14919-s19. Such homokaryons may be suitable for use in the strain development method described immediately above, i.e., a directed paired mating method using two pre-selected, compatible homokaryons to obtain an F1 hybrid strain. In this method, a homokaryon, as a first parent, may be mated with a second homokaryon. It such case, the first parent is a line or homokaryon from Strain B19414 or an EDV of Strain B19414.

In contrast, EDVs are derived directly, solely or predominantly, from a single initial culture (e.g., strain); all such derivations produce EDVs. This definition is congruent with the term as it is widely understood. Methods, i.e. ‘strain development methods,’ of obtaining cultures which are by definition consequently EDVs of a single initial culture of A. bisporus include somatic selection, tissue culture selection, single spore germination, multiple spore germination, selfing, repeated mating back to the initial culture, mutagenesis, and transformation, to provide some examples. DNA-mediated transformation of A. bisporus has been reported by Velcko, A. J. Jr., Kerrigan, R. W., MacDonald, L. A., Wach, M. P., Schlagnhaufer, C., and Romaine, C. P. 2004, Expression of novel genes in Agaricus bisporus using an Agrobacterium-mediated transformation technique. Mush. Sci. 16:591-597, and references therein, herein incorporated by reference. Transformation may introduce a single new gene or allele into the genome of an initial culture. Additionally, recent reports on other closely related fungi raise the possibility of gene editing via CRISPR.

EDVs are unambiguously recognizable by their genotype, which will be predominantly or, more often, entirely a subset of the single initial culture. Percentages of the initial genotype that will be present in Agaricus bisporus EDVs range from 100% or virtually 100% in the case of single spore cultures and of somatic selections, to 99.x % in the case of strains modified by DNA-mediated transformation, to 90-99.x % in the case of some single or multiple spore selections or some mutagenesis, to an average of at least 75-85% in the case of sibling-offspring matings (=selfing) and of back-matings to the initial culture. Many methods of well-known genotype determination, including methods described below, and others well known in the art, may be employed to determine the percentage of DNA of an initial culture that is present in another culture, and to make unambiguous determinations of the relationship between two cultures and any methods used to manipulate or exploit an initial culture.

Repeated mating back to the initial culture also produces an EDV of the initial culture. In a hypothetical example, in the first successive repetition of this process a resultant strain of this generation will have on average about 75% of the DNA of the initial strain while about 25% of the DNA will have been contributed by a second strain or line. As this process is repeated, the DNA representation of the initial strain will increase, to at least 87%. on average, after one further back-mating, to at least 94%, on average, after an additional back-mating, and approaching 97%, on average, after 3 further successive repetitions. It is to be understood that any culture having 75-100% genotype identity with an initial culture is indicative of an EDV of an initial culture, as any culture that descends from (as opposed to being derived from) an initial culture will have only 50% genotype identity with the initial culture. It is also established that an EDV of an EDV is also an EDV of an initial strain, given that both the first EDV and the second EDV will have such a close approximation to the genetic identity of the initial strain. Finally, because Agaricus bisporus alternates generations between heterokaryotic strains and homokaryotic lines, the criteria for essential derivation apply equally to cultures of both strains and lines.

Thus, the present invention further includes a culture of Agaricus bisporus derived directly from an initial culture of strain B19414, wherein a culture of the strain has been deposited under NRRL Accession No. 68095, such that all of its genome or genotype is present in the genome or genotype of the initial culture of strain B19414. In addition, the present invention includes a culture of Agaricus bisporus derived from an initial culture, wherein said initial culture is strain B19414, such that at least 75% of the genome or genotype of the derived culture is present in the genome or genotype of the initial culture of strain B19414, wherein a culture of the strain has been deposited under NRRL Accession No. 68095.

Genotypic fingerprints are descriptions of the genotype at defined loci, where the presence of characterized alleles is recorded. Such fingerprints provide powerful and effective techniques for recognizing clones and all types of EDVs of an initial strain, as well as for recognizing ancestry within outbred lineages. Many techniques are available for defining and characterizing loci and alleles in the genotype. The most detailed approach is provided by whole-genome sequencing (WGS), which allows for direct characterization and comparison of DNA sequences across the entire genome. Using this approach to generate robust genotypic fingerprints incorporating large numbers of marker loci, it is possible to establish the nature of the relationship between two strains, including strains related by genealogical descent over several generations. Applicant has tracked genetic markers through four to six generations of its strain development pedigrees. If a sufficient number of rare markers are present in an initial strain or line, it will be possible to identify descent from an initial strain or line after several outbred generations without undue experimentation. In a hypothetical example, the mean expectation for genomic representation of an initial haploid line after 4 outbred generations is 3.1% (50%/24) in an F4 hybrid, which corresponds to about 1 Mb of the nuclear genomic DNA of A. bisporus. Based on the experimental and mathematical analyses, that amount of DNA from each of two unrelated strains of A. bisporus may typically contain from about 10,000 to about 20,000 Single Nucleotide Polymorphisms (SNPs), any one of which may provide a distinguishing marker linking the F4 hybrid to the initial line. By using multiple independent markers, ancestors of a strain can be identified with a very high probability of success and with reasonable confidence.

As noted above, one trait of biological and commercial interest is heterokaryon incompatibility. The genetics of these self/non-self recognition systems are not well elucidated in basidiomycete fungi such as Agaricus but are known in other groups of fungi to involve multiple alleles at multiple independent loci. Differences in the presumed genotype at the incompatibility loci prevent successful anastomoses and cytoplasmic continuity among physical mixtures of two or more heterokaryons. One consequence of such antagonistic responses is a retardation of growth and development, and a reduction of crop yield; this sort of partial crop failure is well known and evident to the experienced grower. Another consequence of heterokaryon incompatibility is restriction on the opportunity for endocellular viruses to move freely throughout or among mycelial networks. Virus diseases such as those caused by the La France or MVX viruses can have severe negative impacts on facility productivity and must be remediated using hygiene practices which can be assisted by strain rotation. A method of improving mushroom farm hygiene called ‘virus-breaking’ is carried out by replacing cropping material (compost, spawn, casing inoculum) incorporating an initial strain with inoculum and cropping material incorporating another different strain that is incompatible with the initial strain. In the most effective implementation of the virus-breaking method, all biological material of the initial strain at a mushroom farm is replaced with biological material of the second, incompatible strain. Strain incompatibility creates an effective if not absolute barrier to movement of virus from biological reservoirs within a facility into new crops. Rotating cultivation usage among mushroom strains of different genotypes may also interrupt infection and infestation cycles of exogenous pests and pathogens.

Strains currently available to the mushroom industry allow growers to produce crops of mushrooms successfully and usually profitably. Several factors exist that influence the degree of success and profitability achieved. For example, a strain must be able to produce at least a comparable crop yield over two to three breaks or flushes, relative to strains currently being marketed and grown commercially. Also, some physical properties of the mushrooms produced, for example cap color category, and general size and dimensions such as cap diameter, allow mushrooms to be marketed in familiar product categories.

Thus, another aspect of the present invention includes providing a culture that produces a crop of first break portobello mushrooms having brown caps, and having a cap diameter wider than the cap diameter of a cultivar strain selected from the group consisting Heirloom, Tuscan-820, Tuscan-860 and Brawn, at the same stage of maturity. The cultures may also produce a crop of mushrooms having brown caps and having a Flesh Thickness ratio less than 0.49.

In contrast, certain traits, when improved, may confer a higher market value upon mushrooms and/or upon the strain that produces them. Examples of such traits include the shape of the cap, the proportion of less-desirable stem tissue in the mushrooms.

Existing portobello style mushrooms having a relatively rounded cap shape at maturity are suitable for certain culinary preparations (e.g., stuffed mushroom caps), whereas mushrooms with a Flatter Cap Shape are more amenable to popular treatments such as grilling and preparation as a traditional “breakfast flat” meal item and would be more desirable for such uses. Mushrooms having a reduced proportion of stem tissue will be more highly valued by the consumer, as there may be less trim waste, which is an important consideration when mushrooms are sold by weight. Thus, mushrooms and a mushroom strain may have all of the aforementioned improvements while still conforming to the established portobello commercial product category.

Strain B19414 has been shown to produce a crop of mushrooms which have brown caps and have a three break yield at least equivalent to that of an accepted commercial strain, specifically the Heirloom strain, a representative culture of the Heirloom strain having been deposited as BR06 under ATCC Accession No PTA-6876 (U.S. Pat. No. 7,608,760). Strain B19414 produces mushrooms having thinner Flesh Thickness and Cap Flatness at maturity, or have a smaller proportion of stem tissue, compared to Heirloom. More particularly, strain B19414 has a brown cap and has a 3-break crop yield not less than strain Heirloom, and a cap diameter at least as long as (i.e., not significantly different from) cultivar strain Heirloom at the same stage of maturity. Strain B19414, while having the foregoing attributes, also has genetic novelty to enhance crop diversification. Thus, strain B19414 is believed to meet the needs of the market and solves the current problem of the unavailability of such a strain to the market. In addition, strain B19414 differs genotypically when compared to other current brown cultivars (see Tables I to IV below).

These and other advantages of the present invention over existing prior art relating to Agaricus bisporus mushrooms and cultures, which shall become apparent from the description which follows, are accomplished by the invention as hereinafter described and claimed.

One or more aspects of the present invention may be accomplished by a hybrid mushroom culture of Agaricus bisporus designated as strain B19414, a representative culture of the strain having been deposited under NRRL Accession No. 68095. The strain B19414 may include various parts of the culture, including hyphae, spores, and cells and parts of cells, including, nuclei, mitochondria, protoplasts, cell membranes and cell walls, said parts being present in both the vegetative mycelium of the culture and in mushrooms produced by the culture. The spores may be dormant or germinated spores and may include heterokaryons and homokaryons incorporated therein.

One or more products incorporating the hybrid mushroom culture of Agaricus bisporus designated as strain B19414 may be produced. Such products include mycelium, spawn, inoculum, casing inoculum, fresh mushrooms, processed mushrooms, mushroom extracts and fractions, mushroom pieces, and colonized substrates selected from grain, compost, and friable particulate matter. It will be appreciated that mushroom pieces refer to stems, pilei, and other larger portions of the mushroom itself. Spores of the mushrooms may be dormant spores or germinated spores and may include heterokaryons and homokaryons incorporated therein.

One or more other aspects of the present invention may be accomplished by an Essentially Derived Variety (EDV) of the hybrid mushroom culture of strain B19414. In one or more embodiments, an Agaricus bisporus culture produced by essential derivation has at least one of the essential characteristics of strain B19414, for example the same heterokaryon compatibility phenotype, and/or the further characteristics of cap roundness, flesh thickness, yield performance, and yield timing relative to commercial strains Heirloom, Tuscan-820 and Tuscan 860, wherein a culture of strain B19414 has been deposited under the NRRL Accession No. 68095.

Other aspects of the present invention may be accomplished by a method for producing a hybrid culture of Agaricus bisporus that includes a step of mating B12998-s181 (deposited under NRRL Accession Number No. 68093) with a second homokaryon, namely, line P2-s203, a culture of which was deposited under NRRL Accession Number No. 68094. Such a mating provides the mushroom culture B19414, which demonstrates antagonism to a panel of commercial cultivars; Heirloom, Tuscan-820, Tuscan-860, and Brawn. This antagonism demonstrates the genetic distinctness of strain B19414. In one or more embodiments, the method further includes providing a mushroom culture of the invention consisting of mycelium, spawn, inoculum, casing inoculum, fresh mushrooms, process mushrooms, parts of mushrooms, mushroom extracts and fractions, mushroom pieces, and colonized substrates selected from grain, compost, and friable particulate matter. In other embodiments, the method may include providing the mushroom culture in derived cultures selected from the group consisting of, homokaryons, heterokaryons, aneuploids, somatic subcultures, tissue explant cultures, protoplasts, dormant spores, germinating spores, inbred descendants and outbred descendants, transgenic cultures, gene-edited cultures, and cultures having genomes with a single locus conversion.

Still further aspects of the present invention may be accomplished by a hybrid mushroom culture of Agaricus bisporus having a genotypic fingerprint which has characters at marker loci ITS, p1n150-G3-2, MFPC-1-ELF, AN, AF, and FF, wherein all of the characters of said fingerprint are present in the genotypic fingerprint of strain B19414. In one or more embodiments, the culture has a genotypic fingerprint having characters at marker loci described in Table III wherein all of the characters of said fingerprint are present in the genotypic fingerprint of strain B19414.

One or more further aspects of the present invention may be accomplished by a culture, a cell or a culture including the cell, produced by the method(s) above. Thus, one or more embodiments may include a method further including the step of growing the hybrid mushroom culture to produce hybrid mushrooms and parts of mushrooms. Other embodiments may provide for methods wherein the hybrid mushroom culture produced, or the cell, includes a marker profile having characters at marker loci ITS, p1n150-G3-2, MFPC-1-ELF, AN, AF, and FF, wherein all of the characters of said marker profile are also present in the marker profile of strain B19414. Still other embodiments may provide for methods wherein the hybrid mushroom culture produced, or the cell, includes a marker profile having characters at marker loci described in Table I wherein all of the characters of said marker profile are also present in the marker profile of strain B19414.

Finally, another aspect of the present invention may be accomplished by a method that uses the hybrid mushroom culture selected from a strain B19414 or Essentially Derived Varieties of strain B19414, a representative culture of the strain having been deposited under NRRL Accession No. 68095. In one embodiment, the method further includes growing a crop of edible mushrooms by carrying out the steps described hereinabove. In another embodiment, the method may include using strain B19414 or Essentially Derived Varieties of strain B19414 in crop rotation to reduce pathogen pressure and pathogen reservoirs in mushroom growing facilities as described hereinabove. In yet another embodiment, the method includes using strain B19414 and EDVs of strain B19414 to produce homokaryons and offspring as described hereinabove.

### DETAILED DESCRIPTION OF THE INVENTION

Initially, in order to provide clear and consistent understanding of the specification and claims, including the scope to be given such terms, the following definitions are provided.

Allele: One or two or more alternative forms of a gene that arise by mutation and are found at the same place on a chromosome; a heritable unit of the genome at a defined locus, ultimately identified by its DNA sequence (or by other means).

Amphithallism: A reproductive syndrome in which heteromixis and intramixis are both active.

Anastomosis: Fusion of two or more hyphae that achieves cytoplasmic continuity.

Basidiomycete: A monophyletic group of fungi producing meiospores on basidia; a member of a corresponding subdivision of Fungi such as the Basidiomycetales or Basidiomycotina.

Basidium: The meiosporangial cell, in which karyogamy and meiosis occur, and upon which the basidiospores are formed.

Bioefficiency: For mushroom crops, the net fresh weight of the harvested crop divided by the dry weight of the compost substrate at the time of spawning, for any given sampled crop area or compost weight.

Breeding: Development of strains, lines or varieties using methods that emphasize sexual mating.

Cap: Pileus; part of the mushroom, the gill-bearing structure.

Cap Flatness: A measure of the shape or thickness of a mature open mushroom cap.

Cap Roundness: Strictly, a ratio of the maximum distance between the uppermost and lowermost parts of the cap, divided by the maximum distance across the cap, measured on a longitudinally bisected mushroom; typically averaged over many specimens; subjectively, a ‘rounded’ property of the shape of the cap.

Carrier substrate: A medium having both nutritional and physical properties suitable for achieving both growth and dispersal of a culture; examples are substrates that are formulated for mushroom spawn, casing inoculum, and other inoculum.

Casing layer, casing: A layer of non-nutritive material such as peat or soil that is applied to the upper surface of a mass of colonized compost in order to permit development of the mushroom crop.

Casing inoculum (CI): A formulation of inoculum material incorporating a mushroom culture, typically of a defined heterokaryotic strain, suitable for mixing into the casing layer.

Cloning: Somatic propagation without selection.

Combining ability: The capacity of an individual to transmit superior performance to its offspring. General combining ability is an average performance of an individual in a particular series of matings.

Compatibility: See heterokaryon compatibility, vegetative compatibility, sexual compatibility; incompatibility is the opposite of compatibility.

CRISPR: (Clustered Regularly Interspaced Short Palindromic Repeats) A technique in genetic engineering whereby genomes of living organisms can be modified.

Culture: The tangible living organism; the organism propagated on various growth media and substrates; a portion of, or the entirety of one physical strain, line, homokaryon or heterokaryon; the sum of all of the parts of the culture, including hyphae, mushrooms, spores, cells, nuclei, mitochondria, protoplasts, cell membranes and cell walls.

Cultivar: Commercially cultivated variety, or strain

Derivation: Development or obtention of a culture solely or predominantly from an initial strain or culture; see Essentially Derived Variety. The terms ‘derive’ and ‘derived’ refer to this process or to its outcome.

Derived lineage group: The set of Essentially Derived Varieties derived from a single initial strain, and including the initial strain.

Descent: Genealogical descent over a limited number (e.g., 10 or fewer) of generations.

Diploid: Having two haploid chromosomal complements within a single nuclear envelope.

Directed mutagenesis: A process of altering the DNA sequence of at least one specific gene locus.

Essentially Derived Variety (EDV): A culture derived solely or predominantly from an initial strain or culture; a culture that has 75% or more of its genotype present in the genotype of an initial strain, that condition being a consequence of its derivation.

Flesh Thickness: A ratio of the maximum distance between the top of the stem and the uppermost part of the cap, divided by the maximum distance across the cap, measured on a longitudinally bisected mushroom; typically averaged over many specimens; subjectively called ‘meatiness’.

Flush: A period of mushroom production within a cropping cycle, separated by intervals of non-production; the term flush encompasses the terms ‘break’ and ‘wave’ and can be read as either of those terms.

Fungus: A microorganism classified as a member of the Kingdom Fungi.

Gene editing: The process of changing a specific gene, typically via CRISPR-Cas9 or a similar enzyme system, wherein the sequence of a functional gene is changed to make it inactive. In other uses, new sequences (including genes) may be introduced to the genome.

Genealogical relationship: A familial relationship of descent from one or more progenitors, for example that between parents and offspring.

Genetic identity: The genetic information that distinguishes an individual, including representations of said genetic information such as, and including: genotype, genotypic fingerprint, genome sequence, genetic marker profile; “genetically identical”=100% genetic identity. “X % genetically identical”=having X % genetic identity etc.

Genotypic fingerprint: A description of the genotype at a defined set of marker loci; the known genotype.

Gill: Lamella; part of the mushroom, the hymenophore- and basidium-bearing structure.

Haploid: Having only a single complement of nuclear chromosomes; see homokaryon.

Heteroallelic: Having two different alleles at a locus; analogous to heterozygous.

Heteroallelism: Differences between homologous chromosomes in a heterokaryotic genotype; analogous to heterozygosity.

Heterokaryon: As a term of art this refers to a sexual heterokaryon: a culture which has two complementary (i.e., necessarily heteroallelic at the Mat locus) types of haploid nuclei in a common cytoplasm, and is thus functionally and physiologically analogous to a diploid individual (but cytogenetically represented as N+N rather than 2N), and which is reproductively competent (in the absence of any rare interfering genetic defects at loci other than Mat), and which exhibits vegetative incompatibility reactions with other heterokaryons; also called a strain or stock in the strain development context.

Heterokaryon compatibility: The absence of antagonism observed during physical proximity or contact between two heterokaryons that are not genetically identical; see Heterokaryon Incompatibility.

Heterokaryon incompatibility: The phenomenon of antagonism observed during physical proximity or contact between two heterokaryons that are not genetically identical; a multilocus self/non-self recognition system; i.e., a genetic system that allows one heterokaryon culture to discriminate and recognize another culture as being either self or non-self, that operates in basidiomycete heterokaryons to limit anastomosis (hyphal fusion) and cytoplasmic contact; vegetative incompatibility.

Heterokaryotic: Having the character of a heterokaryon.

Heteromixis: Life cycle involving mating between two different non-sibling haploid individuals or gametes; analogous to outbreeding.

Homoallelic: Having not more than one allele at a locus. The equivalent term in a diploid organism is ‘homozygous’. Haploid lines are by definition entirely homoallelic at all non-duplicated loci.

Homokaryon: A haploid culture with a single type (or somatic lineage) of haploid nucleus (cytogenetically represented as N), and which is ordinarily reproductively incompetent, and which does not exhibit typical self/non-self incompatibility reactions with heterokaryons, and which may function as a gamete in sexually complementary anastomoses; a ‘line’ which, as with an inbred plant line, transmits a uniform genotype to offspring; a predominantly homoallelic line that mates well and fruits poorly is a putative homokaryon for strain development purposes; see discussion below.

Homokaryotic: Having the character of a homokaryon; haploid.

Hybrid: Of biparental origin, usually applied to heterokaryotic strains and cultures produced in controlled matings.

Hybridizing: Physical association, for example on a petri dish containing a sterile agar-based nutrient medium, of two cultures, usually homokaryons, in an attempt to achieve anastomosis, plasmogamy, and formation of a sexual heterokaryon (=mating); succeeding in the foregoing.

Hyphae: Threadlike elements of mycelium, composed of cell-like compartments.

Inbreeding: Matings that include sibling-line matings, back-matings to parent lines or strains, and intramixis; reproduction involving parents that are genetically related.

Incompatibility: See heterokaryon incompatibility.

Induced mutagenesis: A non-spontaneous process of altering the DNA sequence of at least one gene locus.

Initial culture: A culture which is used as starting material in a strain development process; more particularly a strain from which an Essentially Derived Variety is obtained.

Inoculum: A culture in a form that permits transmission and propagation of the culture, for example onto new media; specialized commercial types of inoculum include spawn and CI.

Intramixis: A uniparental sexual life cycle involving formation of a complementary ‘mated’ pair of postmeiotic nuclei within the basidium or individual spore.

Lamella: see ‘gill’.

Line: A culture used in matings to produce a hybrid strain; ordinarily a homokaryon which is thus homoallelic, otherwise a non-heterokaryotic (non-NSNPP) culture which is highly homoallelic; practically, a functionally homokaryotic and entirely or predominantly homoallelic culture; analogous in plant breeding to an inbred line which is predominantly or entirely homozygous.

Lineage group: see ‘derived lineage group’. The set of EDVs derived from a single initial strain or variety.

Locus: A defined contiguous part of the genome, homologous although often varying among different genotypes; plural: loci.

Marker assisted selection: Using linked genetic markers including molecular markers to track trait-determining loci of interest among offspring and through pedigrees.

MAT: The mating-type locus, which determines sexual compatibility and the heterokaryotic state.

Mating: The sexual union of two cultures via anastomosis and plasmogamy; methods of obtaining matings between mushroom cultures are well known in the art.

Mycelium: The vegetative body or thallus of the mushroom organism, comprised of threadlike hyphae.

Mushroom: The reproductive structure of an agaric fungus; an agaric; a cultivated food product of the same name.

Neohaplont: A haploid culture or line obtained by physically deheterokaryotizing (reducing to haploid components) a heterokaryon; a somatically obtained homokaryon.

Offspring: Descendants, for example of a parent heterokaryon, within a single generation; most often used to describe cultures obtained from spores from a mushroom of a strain.

Outbreeding: Mating among unrelated or distantly related individuals; analogous to heteromixis in mushrooms.

Parent: An immediate progenitor of an individual; a parent strain is a heterokaryon, a parent line is a homokaryon; a heterokaryon may be the parent of an F1 heterokaryon via an intermediate parent line.

Pedigree-assisted strain development: The use of genealogical information to identify desirable combinations of lines in controlled mating programs.

Phenotype: Observable characteristics of a strain or line as expressed and manifested in an environment.

Plasmogamy: Establishment, via anastomosis, of cytoplasmic continuity leading to the formation of a sexual heterokaryon.

Progenitor: Ancestor, including parent (the direct progenitor).

Selfing: Mating among sibling lines; also intramixis.

Sexual compatibility: A condition among different lines of allelic non-identity at the Mat locus, such that two lines are able to mate to produce a stable and reproductively competent heterokaryon. The opposite condition, sexual incompatibility, occurs when two lines have the same allele at the Mat locus.

Single Nucleotide Polymorphism (SNP): A marker, with a locus position, that varies among individuals and can differentiate two sequences or individuals.

Somatic: Of the vegetative mycelium.

Spawn: A mushroom culture, typically a pure culture of a heterokaryon, typically on a sterile substrate which is friable and dispersible particulate matter, in some instances cereal grain; commercial inoculum for compost; reference to spawn includes reference to the culture on a substrate.

Spore: Part of the mushroom, the reproductive propagule.

Stem: Stipe; part of the mushroom, the cap-supporting structure.

Sterile Growth Media: Nutrient media, sterilized by autoclaving or other methods, that support the growth of the organism; examples include agar-based solid nutrient media such as Potato Dextrose Agar (PDA), nutrient broth, and many other materials.

Stipe: see ‘stem’.

Strain: A heterokaryon with defined characteristics or a specific identity or ancestry; analogous to a variety.

Targeted mutagenesis: A process of altering the DNA sequence of at least one specific gene locus.

Tissue culture: A de-differentiated vegetative mycelium obtained from a differentiated tissue of the mushroom.

Trait conversion: A method for the selective introduction of the genetic determinants of one (a single-locus conversion) or more desirable traits into the genetic background of an initial strain while retaining most of the genetic background of the initial strain.

Transformation: A process by which the genetic material carried by an individual cell is altered by the incorporation of foreign (exogenous) DNA into its genome; a method of obtaining a trait conversion including a single-locus conversion.

Vegetative compatibility: The absence of the phenomenon of antagonism observed during physical proximity or contact between two heterokaryons that are not genetically identical, determined by a multilocus self/non-self recognition system that operates in basidiomycete heterokaryons to limit anastomosis (hyphal fusion) and cytoplasmic contact; Heterokaryon compatibility; the opposite of Vegetative incompatibility.

Vegetative incompatibility: The phenomenon of antagonism observed during physical proximity or contact between two heterokaryons that are not genetically identical, determined by a multilocus self/non-self recognition system that operates in basidiomycete heterokaryons to limit anastomosis (hyphal fusion) and cytoplasmic contact; Heterokaryon incompatibility.

Virus-breaking: Using multiple incompatible strains, i.e. strains exhibiting heterokaryon incompatibility, successively in a program of planned strain rotation within a mushroom production facility to reduce the transmission of virus from on-site virus reservoirs into newly planted crops.

Whole Genome Sequence (WGS): The DNA sequence of an organism, such as Agaricus

Yield: The net fresh weight of the harvest crop, normally expressed in pounds per square foot.

Yield pattern: The distribution of yield within each flush and among all flushes; influences size, quality, picking costs, and relative disease pressure on the crop and product.

With respect to the definition of homokaryon above, it is noted that homokaryons and homoallelic lines are subject to technical and practical considerations: A homokaryon in classical terms is a haploid culture which is axiomatically entirely homoallelic. In practical terms, for fungal strain development purposes, the definition is broadened somewhat to accommodate both technical limitations and cytological variation, by treating all predominately homoallelic lines as homokaryons. Technical limitations include the fact that genomes contain duplicated DNA regions including repeated elements such as transposons and may also include large duplications of chromosomal segments due to historical translocation events; such regions may appear not to be homoallelic by most genotyping methods. Two different A. bisporus genomes sequenced by the Joint Genome Institute, a U.S. federal facility, differ in estimated length by 4.4%, and in gene numbers by 8.2%, suggesting a considerable amount of DNA duplication or rearrangement within different strains of the species. No presently available genome of A. bisporus can completely account for the physical arrangement of such elements and translocations, and so the assembled genome sequences of haploid lines may have regions that appear to be heteroallelic using currently available genotyping methods. Cytologically, a homokaryotic offspring will ordinarily be a spore that receives one haploid, postmeiotic nucleus. However, a spore receiving two third-division nuclei from the basidium will be genetically equivalent to a homokaryon. A spore receiving two second-division ‘sister’ postmeiotic nuclei will be a functional homokaryon even though some distal ‘islands’ of heteroallelism may be present due to crossovers during meiosis. Also, a meiosis that has an asymmetrical separation of homologues can produce an aneuploid, functionally homokaryotic spore in which an extra chromosome, producing a region of heteroallelism, is present. All of these cultures are highly homoallelic and all function as homokaryons. Technological limitations make it impractical to distinguish among such cultures, and also to rule out DNA segment duplication as an explanation for limited, isolated regions of the genome sequence assembly that appear to be heteroallelic. Therefore, in the present application, the use of the term ‘homoallelic’ to characterize a line includes entirely or predominately homoallelic lines, and cultures described in this way are functional homokaryons, are putatively homokaryotic, and are all defined as homokaryons in the present application.

Now, with respect to the invention and as noted hereinabove, the present invention relates to cultures of the hybrid Agaricus bisporus strain B19414 and to cultures derived or descended from strain B19414. Such cultures are used to produce mushrooms and parts of mushrooms. Thus, the present invention further relates to methods of making and using the strain B19414 and Essentially Derived Varieties (EDVs) of the strain B19414.

The morphological and physiological characteristics of strain B19414 in culture on Difco brand PDA medium, which is a standard culture medium, are provided as follows. Strain B19414 growing on PDA medium in an 8.5 cm diameter Petri dish produced a white or light brown-yellow or ‘tan’ colored or darker brown colored irregularly lobate colony with a roughly circular overall outline that increased in diameter by (0.79-0.88-) 0.92 (−0.93-1.11) mm/day during dynamic equilibrium-state growth between days 14 and 24 after inoculation using a 3.5 mm diameter circular plug of the culture on PDA as inoculum. The strain has been increased by transfer of pure inocula into larger volumes of sterile culture media. No variant traits have been observed or are expected in strain B19414.

Hybrid strain B19414 is the product of two generations of controlled line matings by Applicant. To obtain strain B19414, homokaryotic lines derived from spores of each heterokaryotic parent were selected and mated. B12998-s181 and P2-s203 are the two mated lines. The resulting hybrid culture, or heterokaryon, was designated strain B19414.

One use of the culture of strain B19414 is the production of crops of edible mushrooms for sale. Another use is for the improvement of facility hygiene via strain rotation and a ‘virus-breaking’ effect; a related use is for improving production crop diversification. A third use is to incorporate the genetic material of strain B19414 into offspring and derived or descended cultures including dormant and germinating spores and protoplasts. Additional uses also exist as noted above.

Hybridization of Agaricus bisporus cultures of the invention may be accomplished by allowing two different cultures, one of which is a genetic line present in a spore of B19414, to grow together in close proximity, preferably on sterile media, until anastomosis (i.e., hyphal or cell fusion) occurs. In a successful mating, the resultant fusion culture is a first-generation outbred hybrid culture incorporating a genetic line present in a mushroom spore which is one part of one embodiment of the present invention. Protoplasts derived from basidia, or other parts of the organism, are another part of the B19414 mushroom that may be used to transmit genetic material of B19414 into new cultures.

Methods for obtaining, manipulating, and mating cultures of the present invention, for producing offspring, inoculum, products, and crops of the current invention, for using a strain rotation program to improve mushroom farm hygiene, and for obtaining the genotypic fingerprint of mushroom cultures, are described hereinabove, and are also well known to practitioners of the art. Although the invention has been described in terms of particular embodiments in this application, one of ordinary skill in the art, in light of the teachings herein, can generate additional embodiments and modifications without departing from the spirit of, or exceeding the scope of, the claimed invention. Accordingly, it is understood that the descriptions herein are proffered only to facilitate comprehension of the invention and should not be construed to limit the scope thereof.

The four commercial brown controls used in this study represent cultivar cultures that were being grown commercially in Europe, the USA, and other markets in 2020 and 2021; test data were collected by the Assignee of record, Sylvan Inc., in France, the Netherlands, Belgium and the USA. The control strains were Heirloom (Amycel), Tuscan-820 (Sylvan), Tuscan-860 (Sylvan) and Brawn (Amycel). Heirloom was the subject of U.S. Pat. No. 7,608,760, where it was named BRO6 and deposited as ATCC Accession No. PTA-6876. Tuscan-820 is the subject of a pending PCT Application published as WO2022023290A1, where it was named LA3782 and was deposited as CNCM Accession Number 1-5528. Tuscan-860 is known as Tuscan Brown in some sales territories, and was also known as B14528 in U.S. Pat. No. 9,017,988, NRRL Accession number 50900. Brawn is a sister strain to Heirloom and is sold commercially by Amycel in North America. In order to ensure availability, Brawn has been re-deposited by Sylvan in the NRRL under Accession No. 68258.

For the purpose of this invention, the whole genomic DNA sequence of strain B19414 and of the cultures of its parent lines B12998-s181 and P2-s203 have been obtained by the Applicant using the following method. The homokaryotic parent line cultures were grown in sterile broth growth medium after maceration. After 2-4 weeks, hyphal cells were collected by filtration, were frozen at −80 C, and were lyophilized until dry. Cap tissue was obtained from mushrooms produced by cultures of the heterokaryotic B19414 strain and was frozen and lyophilized. DNA was extracted from the lyophilized samples using a CTAB protocol followed by RNAse treatment and gel purification. A contractor. Genewiz (New Brunswick, New Jersey) prepared DNA libraries from the DNA of each culture and sequenced the libraries using Illumina technology. To produce whole genome sequences we utilized Illumina 250 bp reads aligned to the H97 version 2 reference genome using DNAstar version 18. Assemblies of the reads into genomic sequence using the public-domain reference genome sequence of H97 version 2.0 (Morin et al. 2012; PNAS 109 (43): 17501, included herein as a reference) was performed by the Applicant. Consequently about 93% to about 95% of the entire genotype of strain B19414 and its parental homokaryons are known to Sylvan, Inc. with certainty. The reference genome is important because the precise location of each SNP in the genome can be located numerically, with a base pair position known for H97.

In Tables I and II, an SNP-based comparison of the genotypes of the relevant cultures is provided. SNP stands for Single Nucleotide Polymorphism, and represents a marker, with a locus position, that varies among individuals and can differentiate two sequences or individuals.

Data in Tables I and II is displayed as 9-mers and in most cases the middle base (position 5) provides the SNP. Occasionally more than one SNP is present within a 9-mer locus; each such 9-mer is treated as a single SNP. Robust markers at loci aligned across each of the first 19 scaffolds were picked. The SNP alleles at those loci for the homokaryotic parents of B19414 (P2-s203 and B12998-s181) were determined in Table I, while the other four commercial strains included as a comparison (Tuscan-820, Heirloom, Tuscan-860 and Brawn) were determined in Table II.

TABLE I
SNP markers in a panel of inventive strains of brown breeding stocks

| | | | Strains | | |
|---|---|---|---|---|---|
| Scaffold | Ref Pos | H97 2.0 | P2-s203 | B12998-s181 | B19414 |
| scaffold_1 | 99995 | CTACATTGA | CTACGTTGA | CTACGTTGA | CTACGTTGA |
| scaffold_1 | 101993 | GAAGGACAT | GAAGAACAT | GAAGAACAT | GAAGAACAT |
| scaffold_1 | 349966 | AAGGTGGTT | AAGGCGGTT | AAGGCGGTT | AAGGCGGTT |
| scaffold_1 | 660050 | TCACCATGA | TCACTATGA | TCACAATGA | TCACwATGA |
| scaffold_1 | 849951 | GATGGAGGA | GATGAAGGA | GATGGAGGA | GATGrAGGA |
| scaffold_1 | 850014 | ATTCCTTTT | ATTCTTTTT | ATTCTTTTT | ATTCTTTTT |
| scaffold_1 | 867820 | GTCACTATT | GTCACTATT | GTCACTATT | GTCACTATT |
| scaffold_1 | 867860 | ATTCTAAAC | ATTCCAAAC | ATTCCAAAC | ATTCCAAAC |
| scaffold_1 | 867868 | CCTTTCCCA | CCTTTCCCA | CCTTTCCCA | CCTTTCCCA |
| scaffold_1 | 867923 | ATCCAGATG | ATCCAGATG | ATCCGGATG | ATCCrGATG |
| scaffold_1 | 867914 | AAAGCATCG | AAAGGATCG | AAAGGATCG | AAAGGATCG |
| scaffold_1 | 867967 | TCAACTGGT | TCGACTGGy | TCGACTGGy | TCGACTGGy |
| scaffold_1 | 1099971 | GTCGACACC | GTCGGCACC | GTCGACACC | GTCGrCACC |
| scaffold_1 | 1353901 | AGATAACTA | AGATGACTA | AGATGACTA | AGATGACTA |
| scaffold_1 | 1599956 | AATAAGCGC | AATAGGCGC | AATAAGCGC | AATArGCGC |
| scaffold_1 | 1850032 | CGAGTAATT | CGAGCAATT | CGAGCAATT | CGAGCAATT |
| scaffold_1 | 2119049 | ACAATCCAA | ACAACTCAA | ACAACTCAA | ACAACTCAA |
| scaffold_1 | 2401751 | CGGATAAAT | CGGATAAAT | CGGAAAAAT | CGGAwAAAT |
| scaffold_1 | 2635654 | TGCGGTTTG | TGCGATTTG | TGCGATTTG | TGCGATTTG |
| scaffold_1 | 2804522 | GAAGACGAC | GAAGGGGAC | GAAGGGGAC | GAAGGGGAC |
| scaffold_1 | 2858975 | GCCGTTCTT | GCCGCTCTT | GCCGCTCTT | GCCGCTCTT |
| scaffold_1 | 3256057 | TATCTGTTT | TATCCGTTT | TATCCGTTT | TATCCGTTT |
| scaffold_2 | 101820 | ATTAAAGAT | ATTAAAGAT | ATTAAAGAT | ATTAAAGAT |
| scaffold_2 | 128192 | TGGACCAGG | TAGACCCAGG | TGGAAAAGG | TrGAmmAGG |
| scaffold_2 | 279652 | AAGGCATGT | AAGGCATGT | AAGGCATGT | AAGGCATGT |
| scaffold_2 | 350156 | TCGGGGGTG | TCGGGGGTG | TCGGAGGTG | TCGGrGGTG |
| scaffold_2 | 450323 | CTACCCTTG | CTACCCTTG | CTACCCTTG | CTACCCTTG |
| scaffold_2 | 600112 | ATGTATACG | ATGTATACG | ATGTGTACG | ATGTrTACG |
| scaffold_2 | 850338 | TGGTGCTAA | TGGTGCTAA | TGGTTCTAA | TGGTkCTAA |
| scaffold_2 | 1099413 | CCTGACTCA | CCTGACTCA | CCTGGCTCA | CCTGrCTCA |
| scaffold_2 | 1189976 | ACGGCCCAA | ACGGCCCAA | ACGGTCCAA | ACGGyCCAA |
| scaffold_2 | 1293936 | GTGTTTGTT | GTGTTTGTT | GTGTGTGTT | GTGTkTGTT |
| scaffold_2 | 1349512 | CTCAGCAGT | CTCAGCAGT | CTCAACAGT | CTCArCAGT |
| scaffold_2 | 1378074 | TCCACTTCA | TCCACTTCA | TCCATTTCA | TCCAyTTCA |
| scaffold_2 | 1378104 | TTTCCAGAT | TTTCCAGAT | TTCCTAGAT | TTyCyAGAT |
| scaffold_2 | 1600085 | CACAATGCC | CACAATGCC | CACATTGCC | CACAwTGCC |
| scaffold_2 | 1643101 | CATCTTCTT | CATCCTCTT | CATCGTCTT | CATCsTCTT |
| scaffold_2 | 2150162 | TGCTTAGGG | TGCTTAGGG | TGCTGAGGG | TGCTkAGGG |
| scaffold_2 | 2389428 | GGATTTCAA | GGATGTCAA | GGATGTCAA | GGATGTCAA |
| scaffold_2 | 2400281 | TCAAAACCC | TCAACACTC | CCAACACCC | yCAACACyC |
| scaffold_2 | 2650136 | ATAATTCCT | ATAATTCCT | ATAAGTCCT | ATAAkTCCT |
| scaffold_2 | 2904101 | TGTTGAGGT | TGTTGAGGT | TGTTAAGGT | TGTTrAGGT |
| scaffold_2 | 3049515 | GAAAAGCTT | GAAAGGCTT | GAAAGGCTT | GAAArGCTT |
| scaffold_3 | 57118 | TATAGCAGC | TATAGCAGC | TATGACAGC | TATrrCAGC |
| scaffold_3 | 118150 | GTTTGTCCT | GTTTGTCCT | GTTTATCCT | GTTTrTCCT |
| scaffold_3 | 131389 | AGACCGGCG | AGACCGGCG | AGACCGGCG | AGACCGGCG |
| scaffold_3 | 175472 | CTTTATTTC | CTTTATTTC | CTTTGTTTC | CTTTrTTTC |
| scaffold_3 | 250112 | GCAGGAGAG | GCAGGAGAG | GCCGAAGAG | GCmGrAGAG |
| scaffold_3 | 379203 | ATAGCGGAA | ATAGCGGAA | ATAGTGGAA | ATAGyGGAA |
| scaffold_3 | 614937 | CAAAATCTG | CAAAATCGT | CAAACTCGT | CAAAmTCGT |
| scaffold_3 | 750074 | GTTCTTTTC | GTTCTTTTC | GTTCATTTC | GTTCwTTTC |
| scaffold_3 | 1126997 | TCAAAGGCG | TCAAAGGCG | TCAAGGGCG | TCAArGGCG |
| scaffold_3 | 1250161 | AGTCTCCTT | AGTCTCCTT | AGTCCCCTT | AGTCyCCTT |
| scaffold_3 | 1296141 | ATCGGTCAT | ATCGGTCAT | ATCGTTCAT | ATCGkTCAT |
| scaffold_3 | 1510819 | CCACTGATT | CCACCGATT | CCACTGATT | CCACyGATT |
| scaffold_3 | 1774892 | CCGTATGGG | CCGTATGGG | CCGTCTGGG | CCGTmTGGG |
| scaffold_3 | 2008438 | AGCATAGCC | AGCATAGCC | AGCAAAGCC | AGCAwAGCC |
| scaffold_3 | 2250000 | CGTGGCGAT | CGTGGCGAT | CGTGGCGAT | CGTGGCGAT |
| scaffold_3 | 2274053 | AAACCAAGA | AAACCAAGA | AAACAAAGA | AAACmAAGA |
| scaffold_3 | 2384173 | TGACCAAGC | TGACCAAGC | TGACAAAGC | TGACmAAGC |
| scaffold_3 | 2520748 | TAATTCCAC | TAATTCCAC | TAATTCCAC | TAATTCCAC |
| scaffold_3 | 2523207 | CAGTCCATA | CAGTCCATA | CAGTCCATA | CAGTCCATA |
| scaffold_4 | 100004 | GAGTGATAA | GAGTGATAA | GAGTGATAA | GAGTGATAA |
| scaffold_4 | 460303 | TCCTATAAC | TCCTATAAC | TCCTGTAAC | TCCTrTAAC |
| scaffold_4 | 490648 | CGATCGCGT | CGATCGCGT | CGATCGCGT | CGATCGCGT |
| scaffold_4 | 649317 | GAGGCAATG | GAGGCAATG | GAGGTAATA | GAGGyAATr |
| scaffold_4 | 752893 | AAGTCCCAA | AAGTCCCAA | AAGTTCCAA | AAGTyCCAA |
| scaffold_4 | 753018 | TGGGCAAGC | TGGGCAAGC | TGGGAAAGC | TGGGmAAGC |
| scaffold_4 | 753116 | — | — | GATATC | gatatc |
| scaffold_4 | 753134 | AACATAACT | AACATAACT | AACAGAACT | AACAkAACT |
| scaffold_4 | 753221 | CTGTTGGAC | CTGTTGGAC | CTGTCGGAC | CTGTyGGAC |
| scaffold_4 | 878926 | CTGATCAAT | CTGATCAAT | CCGACCAAT | CyGAyCAAT |
| scaffold_4 | 1100085 | GATGCCGAA | GATGCCGAA | GATGACGAA | GATGmCGAA |
| scaffold_4 | 1163185 | CAAGCTACT | CAAGCTACT | CAAAATACT | CAArmTACT |
| scaffold_4 | 1350536 | CGAACTCGG | CGAACTCGG | CGAAACCGG | CGAAmyCGG |
| scaffold_4 | 1599885 | GATACTTGC | GATACTTGC | GATAATTGC | GATAmTTGC |
| scaffold_4 | 1850288 | ATTCGTGTA | ATTCGTGTA | ATTCACGTA | ATTCryGTA |
| scaffold_4 | 1889549 | ACAACAGAA | ACAACAGAA | ACAAAAGAA | ACAAmAGAA |
| scaffold_4 | 2100356 | TCAGAGACC | TCAGGGACC | TCAGAGACC | TCAGrGACC |
| scaffold_4 | 2284257 | TCTGGACTG | TCTGAACTG | TCTGGACTG | TCTGrACTG |
| scaffold_5 | 87962 | GATTAAGGG | GATTGAGGG | GATTAAGGG | GATTrAGGG |
| scaffold_5 | 100211 | TCCTTGAAT | TCCTCGAAT | TCCTCGAAT | TCCTCGAAT |
| scaffold_5 | 350872 | GGCGTGCCC | GGCGCGCCC | GGCGTGCCC | GGCGyGCCC |
| scaffold_5 | 599922 | CGTCATTCA | CGTCGTTCA | CGTCATTCA | CGTCrTTCA |
| scaffold_5 | 851262 | TAATTCTCT | TAATCGTCT | TAATCGTCT | TAATCGTCT |
| scaffold_5 | 1099776 | ACATTGACA | ACATCGACA | ACATCGACA | ACATCGACA |
| scaffold_5 | 1352539 | TTGTGATCC | TTGTTGTCC | TTGTGATCC | TTGTkrTCC |
| scaffold_5 | 1599904 | AACTTCCTT | AACTCCCTT | AACTCCCTT | AACTCCCTT |
| scaffold_5 | 1851487 | TTCCGCTCC | TTCCGCTCC | TTCCCCTCC | TTCCsCTCC |
| scaffold_5 | 2100025 | CCCTTAGTC | CCCTyAGTC | CCCTyAGTC | CCCTyAGTC |
| scaffold_5 | 2278878 | GGTCGAAAA | GGTCAAAAA | GGTCGAAAA | GGTCrAAAA |
| scaffold_6 | 106480 | GCCCACTTG | GCCCGCTTG | GCCCACTTG | GCCCrCTTG |
| scaffold_6 | 350337 | CATTTGGTT | CATTCGGTT | CATTTGGTT | CATTyGGTT |
| scaffold_6 | 600047 | GGAGCATTT | GGAGTATTT | GGAGCATTT | GGAGyATTT |
| scaffold_6 | 849990 | AGTTCAGGA | AGTTTAGGA | AGTTCAGGA | AGTTyAGGA |
| scaffold_6 | 1098535 | CAAAGATTG | CAAAAATTG | CAAAGATTG | CAAArATTG |
| scaffold_6 | 1349453 | TGTCGGTAG | TGTCAATAG | TGTCGGTAG | TGTCrrTAG |
| scaffold_6 | 1600000 | AAACCTGGA | AAACCTGGA | AAACCTGGA | AAACCTGGA |
| scaffold_6 | 1764645 | AACCGGATT | AACCAGATT | AACCGGATT | AACCrGATT |
| scaffold_6 | 2000087 | GATTTTGCG | GATTTTGCG | GATTCTGCG | GATTYTGCG |
| scaffold_6 | 2007502 | AATTGATAA | AATTGATAA | AATTGATAA | AATTGATAA |
| scaffold_7 | 348994 | CCGGAGTTT | CCGGAGTTT | CCGGCGTTT | CCGGmGTTT |
| scaffold_7 | 600111 | CAATTATTA | CAATTATTA | CAATCATTA | CAATyATTA |
| scaffold_7 | 850516 | TGACGCATA | TGACGCATA | TGACACATA | TGACrCATA |
| scaffold_7 | 873221 | AATAGACCT | AATAGACCT | AATAAACCT | AATArACCT |
| scaffold_7 | 1100248 | TCACGGAAG | TCACGGAAG | TCACAGAAG | TCACrGAAG |
| scaffold_7 | 1352529 | TAAATATAT | TAAATATAT | TAAATATAT | TAAATATAT |
| scaffold_7 | 1605059 | GACAAGCAA | GACAAGCAA | GACAGGCAA | GACArGCAA |
| scaffold_7 | 1991524 | CAACCCACC | CAACCCACC | CAACTCACC | CAACyCACC |
| scaffold_8 | 350000 | ATTGACGCG | ATTGGCGCG | ATTGGCGCG | ATTGGCGCG |
| scaffold_8 | 606991 | GTGTATTCT | GTGTGTTCT | GTGTCTTCT | GTGTsTTCT |
| scaffold_8 | 610549 | GGAACTTGA | GGAATTTGA | GGAATACGA | GGAATwyGA |
| scaffold_8 | 829832 | CTGTACAAC | CTGTACAAC | CTGTGCAAC | CTGTrCAAC |
| scaffold_8 | 829846 | TTCGAGTGA | TTCGAGTGA | TTCGAGTGA | TTCGAGTGA |
| scaffold_8 | 830003 | AACTGGCAG | AACTAGCAG | AACTGGCAG | AACTrGCAG |
| scaffold_8 | 830070 | ATTAGGATT | ATTAGGATT | ATTAGGATT | ATTAGGATT |
| scaffold_8 | 830078 | TACTAGACG | TACTGGACG | TACTGGACG | TACTGGACG |
| scaffold_8 | 830105 | ATTTAGCAT | ATTTAGCAT | ATTTAGCAT | ATTTAGCAT |
| scaffold_8 | 830159 | AATTAGAAG | AATTAGAAG | AATTAGAAG | AATTAGAAG |
| scaffold_8 | 830169 | GACGACTGG | GACGACTGG | GACGACTGG | GACGACTGG |
| scaffold_8 | 830215 | AGTGTATCT | AGTGCATCT | AGTGCATCT | AGTGCATCT |
| scaffold_8 | 830250 | TCCAATGCA | TCCAGTGCA | TCCAGTGCA | TCCAGTGCA |
| scaffold_8 | 1100000 | CATACGATC | CATACGATC | CATACGATC | CATACGATC |
| scaffold_8 | 1350240 | ACGGGTACT | ACGGGTACT | ACGGATACT | ACGGrTACT |
| scaffold_8 | 1354068 | AGAATGCCT | AGAAAGTTT | AGAAAGCCT | AGAAAGyyT |
| scaffold_8 | 1614036 | TTATCAGTA | TTATCAGTA | TTATTAGTA | TTATyAGTA |
| scaffold_8 | 1869238 | TGGAGGTTG | TGGATGTTG | TGGACGTTG | TGGAyGTTG |
| scaffold_9 | 100447 | CTATTTTCT | CTATTTTCT | CTATGTTCT | CTATkTTCT |
| scaffold_9 | 350569 | AGAATATAC | AGAATATAC | AGAATATAC | AGAATATAC |
| scaffold_9 | 599950 | TGGTATCCC | TGGTGTCCC | TGGTGTCCC | TGGTGTCCC |
| scaffold_9 | 611788 | TCTGTAATC | TTTGTAATC | TTTGTAATC | TTTGTAATC |
| scaffold_9 | 721973 | TGTATACGT | TGTATACGT | TGTAGACGT | TGTAkACGT |
| scaffold_9 | 1010845 | GGGTGGTGA | GGGTGGTGA | GGGTGGTGA | GGGTGGTGA |
| scaffold_9 | 1250830 | TTGTGGGGA | TTGTGGGGA | TTGTAGGGA | TTGTrGGGA |
| scaffold_9 | 1499265 | AGTCAGACA | AGTCCGACA | AGTCCGACA | AGTCCGACA |
| scaffold_9 | 1499300 | TATGACACC | TATGACGCC | TATGACGCC | TATGACGCC |
| scaffold_9 | 1676755 | CTGCCGTTT | CTGCCGTTT | CTGCCGTTT | CTGCCGTTT |
| scaffold_9 | 1702348 | AGACGCATC | AGAAACATC | AGACGCATC | AGAmACATC |
| scaffold_9 | 1702552 | CAAAGTCAT | CAAAGTCAT | CAAAGTCAT | CAAAGTCAT |
| scaffold_9 | 1702583 | ACTCAGCTG | ACTCAGCTG | ACTCAGCTG | ACTCAGCTG |
| scaffold_9 | 1702658 | TTGTCGTGG | TTGTCGTGG | TTGTCGTGG | TTGTCGTGG |
| scaffold_10 | 100470 | TCACCATCG | TCACCATCG | TCACCATCG | TCACCATCG |
| scaffold_10 | 350030 | GCGGCTCAA | GCGGTTCAA | GCGGCTCAA | GCGGyTCAA |
| scaffold_10 | 354531 | AATCAATCA | AATCmATCA | AATCmATCA | AATCmATCA |
| scaffold_10 | 633622 | TGGGCAAAG | TGGGGAAAG | TGGGCAAAG | TGGGsAAAG |
| scaffold_10 | 860249 | CCGCAAATT | CCGCAAATT | CCGCAAATT | CCGCAAATT |
| scaffold_10 | 863401 | ATAAAATTT | ATAAAATTT | ATAAAATTT | ATAAAATTT |
| scaffold_10 | 1107782 | CAACCCCAC | poor depth | CAACCCCAC | CAACCCCAC |
| scaffold_10 | 1338596 | GTGCATCAT | GTGCCTCAT | GTGCATCAT | GTGCmTCAT |
| scaffold_10 | 1477092 | AGATGCAAA | AGATGCAAA | AGATGCAAA | AGATGCAAA |
| scaffold_10 | 1612161 | TCTTCGGAG | TCTTTGGAG | TCTTCGGAG | TCTTyGGAG |
| scaffold_10 | 1612569 | ATTATATTC | ATTATATTC | ATTATATTC | ATTATATTC |
| scaffold_10 | 1612630 | TGGCTCCTT | TGGCTCCTT | TGGCTCCTT | TGGCTCCTT |
| scaffold_10 | 1612671 | GGAATCGTC | GGAATCGTC | GGAATCGTC | GGAATCGTC |
| scaffold_11 | 101855 | CCAGCCTGT | CCAGCCTGT | CCAGCCTGT | CCAGCCTGT |
| scaffold_11 | 173230 | AGCGGGCGA | AGCGCGCGA | AGCGGGCGA | AGCGsGCGA |
| scaffold_11 | 350000 | GTCAGCAAG | GTCAGCAAG | GTCAGCAAG | GTCAGCAAG |
| scaffold_11 | 378409 | TGATTGGGG | TGATAGGGG | TGATTGGGG | TGATwGGGG |
| scaffold_11 | 600001 | TGGGCGCGC | TGGGAGCGC | TGGGCGCGC | TGGGmGCGC |
| scaffold_11 | 627221 | TCTTCGCCC | TCTTTGCCC | TCTTCGCCC | TCTTyGCCC |
| scaffold_11 | 929659 | GGAATATCA | GGAAGTTCA | GGAATATCA | GGAAkwTCA |
| scaffold_11 | 931877 | GACCTCACC | GACCGCACC | GACCTCACC | GACCkCACC |
| scaffold_11 | 1155850 | T-TGCCACG | TATCCCACG | T-TGCCACGC | TATsCCACG |
| scaffold_11 | 1240230 | ACAAGATTC | ACAAGATTC | ACAAGATTC | ACAAGATTC |
| scaffold_11 | 1250447 | GAGGCTACA | GAGGATACA | GAGGCTACA | GAGGmTACA |
| scaffold_12 | 109790 | GTCTGCACC | GTCTGCACC | GTCTGCACC | GTCTGCACC |
| scaffold_12 | 272255 | CCGAGTGCT | CCGAGTGCT | CCGAGTGCT | CCGAGTGCT |
| scaffold_12 | 281720 | CTTCCGGCG | CTTCCGGCG | CTTCCGGCG | CTTCCGGCG |
| scaffold_12 | 281763 | TCTGCAGCC | TCTGCAGCC | TCTGCAGCC | TCTGCAGCC |
| scaffold_12 | 554582 | ACTCCGGTC | ACTCCGGTC | ACTCTGGTC | ACTCyGGTC |
| scaffold_12 | 770075 | GAACGTTCT | GAACGTTCT | GAACATTCT | GAACrTTCT |
| scaffold_12 | 909536 | CTATGGAGG | CTATAAAGG | CTATAAAGG | CTATAAAGG |
| scaffold_12 | 1000000 | CGAGGAGGA | CGAGAAGGA | CGAGGAGGA | CGAGrAGGA |
| scaffold_13 | 100697 | ACGTCTTTA | ACGTCTTTA | ACGTCTTTA | ACGTCTTTA |
| scaffold_13 | 119283 | ACGTTACTG | ACGTTACTG | ACGCCACTG | ACGyyACTG |
| scaffold_13 | 363867 | ATCCACTGC | ATCCACTGC | ATCCGCTGC | ATCCrCTGC |
| scaffold_13 | 370521 | TTTGAGTCA | TTTGAGTCA | TTTGTGTCA | TTTGwGTCA |
| scaffold_13 | 604345 | CTTCAGCAT | CTTCAGCAT | CTTCAGCAT | CTTCAGCAT |
| scaffold_13 | 866136 | GTTGGTCAG | GTTGGTCAG | GTTGATCAG | GTTGrTCAG |
| scaffold_14 | 113109 | AGGGAAATA | AGGGAAATA | AGGGGAATA | AGGGrAATA |
| scaffold_14 | 372086 | CGATCCCTT | CGATCCCTT | CGATTCTTT | CGATyCyTT |
| scaffold_14 | 603118 | GGCCCGCCT | GGCCCGCCT | GGCCGGCCT | GGCCsGCCT |
| scaffold_14 | 725687 | AGTTCGAAA | AGTTCGAAA | AGTTTGGAA | AGTTyGrAA |
| scaffold_14 | 808308 | AAGGTATGG | AAGGTATGG | AAGGTATGG | AAGGTATGG |
| scaffold_15 | 101381 | TAAACAGAT | TAAACAGAT | TAAATAGAT | TAAAyAGAT |
| scaffold_15 | 150013 | GTGGCCCGT | GTGGCCCGT | GTGGACCGT | GTGGmCCGT |
| scaffold_15 | 367204 | CGCGCCCTA | CGCGCCCTA | CGCGACCTA | CGCGmCCTA |
| scaffold_16 | 106292 | AAGCTGGAA | AAGCCGGAA | AAGCAGGAA | AAGCmGGAA |
| scaffold_16 | 205778 | CAAGGTCTG | CAAGATCTG | CAAGATCTG | CAAGATCTG |
| scaffold_16 | 400000 | CCTCGGATT | CCTCGGATT | CCTCGGATT | CCTCGGATT |
| scaffold_16 | 403998 | CAAAGTACG | CAAAGTACG | CAAAATACG | CAAArTACG |
| scaffold_17 | 134688 | CCCGCTTCA | CCCGCTTCA | CCCGCTTCA | CCCGCTTCA |
| scaffold_17 | 370858 | GACACAACG | GACAAAACG | GACATAACG | GACAwAACG |
| scaffold_17 | 449833 | ATCAGACAA | ATCAAACTA | ATCAAACTA | ATCAAACTA |
| scaffold_17 | 472545 | CCGTTCATG | CCGTTCATG | CCGTCCGTG | CCGTyCrTG |
| scaffold_18 | 112940 | GCGGGTGGG | GCGGGTGGG | GCGGGTGGG | GCGGGTGGG |
| scaffold_18 | 126322 | CCTCTTCCG | CCTCTTCCG | CCTCGTCCG | CCTCkTCCG |
| scaffold_19 | 87323 | CCCAAGCAA | CCCAAGCAA | CCCACGCAA | CCCAmGCAA |
| scaffold_19 | 98782 | AAAATTGTT | AAAATTGTT | AAAAGTGTT | AAAAkTGTT |

TABLE II
SNP markers in a panel of comparison strains of brown breeding stocks

| | | | Strains | | | |
|---|---|---|---|---|---|---|
| Scaffold | Ref Pos | H97 2.0 | Tuscan-820 | Heirloom | Tuscan-860 | Brawn |
| scaffold_1 | 99995 | CTACATTGA | CTACGTTGA | CTACGTTGA | CTACGTTGA | CTACGTTGA |
| scaffold_1 | 101993 | GAAGGACAT | GAAGAACAT | GAAGrACAT | GAAGAACAT | GAAGrACAT |
| scaffold_1 | 349966 | AAGGTGGTT | AAGGCGGTT | AAGGyGGTT | AAGGCGGTT | AAGGyGGTT |
| scaffold_1 | 660050 | TCACCATGA | TCACwATGA | TCACmATGA | TCACwATGA | TCACmATGA |
| scaffold_1 | 849951 | GATGGAGGA | GATGrAGGA | GATGGAGGA | GATGrAGGA | GATGGAGGA |
| scaffold_1 | 850014 | ATTCCTTTT | ATTCTTTTT | ATTCyTTTT | ATTCTTTTT | ATTCyTTTT |
| scaffold_1 | 867820 | GTCACTATT | GTCACTATT | GTCACTATT | GTCACTATT | GTCACTATT |
| scaffold_1 | 867860 | ATTCTAAAC | ATTCCAAAC | ATTCyAAAC | ATTCCAAAC | ATTCyAAAC |
| scaffold_1 | 867868 | CCTTTCCCA | CCTTTCCCA | CCTTTCCCA | CCTTTCCCA | CCTTTCCCA |
| scaffold_1 | 867923 | ATCCAGATG | ATCCrGATG | ATCCrGATG | ATCCrGATG | ATCCrGATG |
| scaffold_1 | 867914 | AAAGCATCG | AAAGGATCG | AAAGsATCG | AAAGGATCG | AAAGsATCG |
| scaffold_1 | 867967 | TCAACTGGT | TCGACTGGy | TCrACTGGy | TCGACTGGy | TCrACTGGT |
| scaffold_1 | 1099971 | GTCGACACC | GTCGrCACC | GTCGACACC | GTCGrCACC | GTCGACACC |
| scaffold_1 | 1353901 | AGATAACTA | AGATGACTA | AGATrACTA | AGATGACTA | AGATrACTA |
| scaffold_1 | 1599956 | AATAAGCGC | AATArGCGC | AATAAGCGC | AATArGCGC | AATAAGCGC |
| scaffold_1 | 1850032 | CGAGTAATT | CGAGCAATT | CGAGyAATT | CGAGCAATT | CGAGyAATT |
| scaffold_1 | 2119049 | ACAATCCAA | ACAACTCAA | ACAACTCAA | ACAACTCAA | ACAACTCAA |
| scaffold_1 | 2401751 | CGGATAAAT | CGGAwAAAT | CGGAwAAAT | CGGAwAAAT | CGGAwAAAT |
| scaffold_1 | 2635654 | TGCGGTTTG | TGCGATTTG | TGCGATTTG | TGCGATTTG | TGCGATTTG |
| scaffold_1 | 2804522 | GAAGACGAC | GAAGGGGAC | GAAGrsGAC | GAAGGGGAC | GAAGrsGAC |
| scaffold_1 | 2858975 | GCCGTTCTT | GCCGCTCTT | GCCGyTCTT | GCCGCTCTT | GCCGyTCTT |
| scaffold_1 | 3256057 | TATCTGTTT | TATCCGTTT | TATCCGTTT | TATCCGTTT | TATCCGTTT |
| scaffold_2 | 101820 | ATTAAAGAT | ATTAAAGAT | ATTAAAGAT | ATTAAAGAT | ATTAAAGAT |
| scaffold_2 | 128192 | TGGACCAGG | TrGAmmAGG | TGGAwmAGG | TrGAmmAGG | TGGAwmAGG |
| scaffold_2 | 279652 | AAGGCATGT | AAGGCATGT | AAGGyATGT | AAGGCATGT | AAGGyATGT |
| scaffold_2 | 350156 | TCGGGGGTG | TCGGrGGTG | TCGGAGGTG | TCGGrGGTG | TCGGAGGTG |
| scaffold_2 | 450323 | CTACCCTTG | CTACCCTTG | CTACCCTTG | CTACCCTTG | CTACCCTTG |
| scaffold_2 | 600112 | ATGTATACG | ATGTrTACG | ATGTGTACG | ATGTrTACG | ATGTGTACG |
| scaffold_2 | 850338 | TGGTGCTAA | TGGTkCTAA | TGGTGCTAA | TGGTkCTAA | TGGTkCTAA |
| scaffold_2 | 1099413 | CCTGACTCA | CCTGrCTCA | CCTGGCTCA | CCTGrCTCA | CCTGGCTCA |
| scaffold_2 | 1189976 | ACGGCCCAA | ACGGyCCAA | ACGGwCCAA | ACGGyCCAA | ACGGwCCAA |
| scaffold_2 | 1293936 | GTGTTTGTT | GTGTkTGTT | GTGTrTGTT | GTGTkTGTT | GTGTrTGTT |
| scaffold_2 | 1349512 | CTCAGCAGT | CTCArCAGT | CTCAACGGT | CTCArCAGT | CTCArCGGT |
| scaffold_2 | 1378074 | TCCACTTCA | TCCAyTTCA | TCCATTTCA | TCCAyTTCA | TCCATTTCA |
| scaffold_2 | 1378104 | TTTCCAGAT | TTyCyAGAT | TTCCTAGAT | TTyCyAGAT | TTCCTAGAT |
| scaffold_2 | 1600085 | CACAATGCC | CACAwTGCC | CACAATGCC | CACAwTGCC | CACAwTGCC |
| scaffold_2 | 1643101 | CATCTTCTT | CATCsTCTT | CATCTTCTT | CATCsTCTT | CATCTTCTT |
| scaffold_2 | 2150162 | TGCTTAGGG | TGCTkAGGG | TGCTTAGGG | TGCTkAGGG | TGCTkAGGG |
| scaffold_2 | 2389428 | GGATTTCAA | GGATGTCAA | GGATKTCAA | GGATGTCAA | GGATGTCAA |
| scaffold_2 | 2400281 | TCAAAACCC | yCAACACyC | TCAAmACCC | yCAACACyC | TCAACACCC |
| scaffold_2 | 2650136 | ATAATTCCT | ATAAGTCCT | ATAATTCCT | ATAAkTCCT | ATAAkTCCT |
| scaffold_2 | 2904101 | TGTTGAGGT | TGTTrAGGT | TGTTGAGGT | TGTTrAGGT | TGTTrAGGT |
| scaffold_2 | 3049515 | GAAAAGCTT | GAAArGCTT | GAAArGCTT | GAAAGGCTT | GAAAGGCTT |
| scaffold_3 | 57118 | TATAGCAGC | TATrrCAGC | TATrrCAGC | TATAGCAGC | TATrrCAGC |
| scaffold_3 | 118150 | GTTTGTCCT | GTTTrTCCT | poor depth | GTTTGTCCT | GTTTGTCCT |
| scaffold_3 | 131389 | AGACCGGCG | AGACCGGCG | AGACCGGCG | AGACCGGCG | AGACCGGCG |
| scaffold_3 | 175472 | CTTTATTTC | CTTTrTTTC | CTTTrTTTC | CTTTwTTTC | CTTTrTTTC |
| scaffold_3 | 250112 | GCAGGAGAG | GCmGrAGAG | GCmGrAGAG | GCmGrAGAG | GCmGrAGAG |
| scaffold_3 | 379203 | ATAGCGGAA | ATAGyGGAA | ATAGyGGAA | ATAGmGGAA | ATAGCGGAA |
| scaffold_3 | 614937 | CAAAATCTG | CAAAmTCGT | CAAAmTCTG | CAAAwTCrG | CAAAmTCTG |
| scaffold_3 | 750074 | GTTCTTTTC | GTTCwTTTC | GTTCwTTTC | GTTCwTTTC | GTTCwTTTC |
| scaffold_3 | 1126997 | TCAAAGGCG | TCAArGGCG | TCAArGGCG | TCAArGGCG | TCAArGGCG |
| scaffold_3 | 1250161 | AGTCTCCTT | AGTCyCCTT | AGTCyCCTT | AGTCTCCTT | AGTCyCCTT |
| scaffold_3 | 1296141 | ATCGGTCAT | ATCGkTCAT | ATCGkTCAT | ATCGrTCAT | ATCGkTCAT |
| scaffold_3 | 1510819 | CCACTGATT | CCACyGATT | CCACyGATT | CCACwGATT | CCACyGATT |
| scaffold_3 | 1774892 | CCGTATGGG | CCGTmTGGG | CCGTmTGGG | CCGTrTGGG | CCGTATGGG |
| scaffold_3 | 2008438 | AGCATAGCC | AGCAwAGCC | AGCAwAGCC | AGCAkAGCC | AGCATAGCC |
| scaffold_3 | 2250000 | CGTGGCGAT | CGTGGCGAT | CGTGGCGAT | CGTGGCGAT | CGTGGCGAT |
| scaffold_3 | 2274053 | AAACCAAGA | AAACmAAGA | AAACmAAGA | AAACyAAGA | AAACmAAGA |
| scaffold_3 | 2384173 | TGACCAAGC | TGACmAAGC | TGACmAAGC | TGACyAAGC | TGACmAAGC |
| scaffold_3 | 2520748 | TAATTCCAC | TAATkCCAC | TAATkCCAC | TAATTCCAC | TAATkCCAC |
| scaffold_3 | 2523207 | CAGTCCATA | CAGTyyATA | CAGTyyATA | CAGTCCATA | CAGTyyATA |
| scaffold_4 | 100004 | GAGTGATAA | GAGTGATAA | GAGTGATAA | GAGTGATAA | GAGTGATrA |
| scaffold_4 | 460303 | TCCTATAAC | TCCTmTAAC | TCCTmTAAC | TCCTrTAAC | TCCTrTAAC |
| scaffold_4 | 490648 | CGATCGCGT | CGATyGCGT | CGATCGCGT | CGATCGCGT | CGATCGCGT |
| scaffold_4 | 649317 | GAGGCAATG | GAGGyAATr | GAGGCAATG | GAGGyAATr | GAGGCAATG |
| scaffold_4 | 752893 | AAGTCCCAA | AAGTCCCAA | AAGTCCCAA | AAGTyCCAA | AAGTCCCAA |
| scaffold_4 | 753018 | TGGGCAAGC | TGGGmAAGC | TGGGmAAGC | TGGGmAAGC | TGGGmAAGC |
| scaffold_4 | 753116 | — | — | — | gatatc | — |
| scaffold_4 | 753134 | AACATAACT | AACAkAACT | AACAkAACT | AACAkAACT | AACATAACT |
| scaffold_4 | 753221 | CTGTTGGAC | CTGTyGGAC | CTGTyGGAC | CTGTyGGAC | CTGTyGGAC |
| scaffold_4 | 878926 | CTGATCAAT | CyGAyCAAT | CyGAyCAAT | CyGAyCAAT | CyGAyCAAT |
| scaffold_4 | 1100085 | GATGCCGAA | GATGmCGAA | GATGmCGAA | GATGmCGAA | GATGmCGAA |
| scaffold_4 | 1163185 | CAAGCTACT | CAAGyTACT | CAAGyTACT | CAArmTACT | CAAryTACT |
| scaffold_4 | 1350536 | CGAACTCGG | CGAAmyCGG | CGAAmyCGG | CGAAmyCGG | CGAAmyCGG |
| scaffold_4 | 1599885 | GATACTTGC | GATACTTGC | GATACTTGC | GATAmTTGC | GATACTTGC |
| scaffold_4 | 1850288 | ATTCGTGTA | ATTCryGTA | ATTCryGTA | ATTCryGTA | ATTCGTGTA |
| scaffold_4 | 1889549 | ACAACAGAA | ACAAsAGAA | ACAAsAGAA | ACAAmAGAA | ACAAsAGAA |
| scaffold_4 | 2100356 | TCAGAGACC | TCAGrGACC | TCAGrGACC | TCAGAGACC | TCAGrGACC |
| scaffold_4 | 2284257 | TCTGGACTG | TCTGAACTG | TCTGAACTG | TCTGGACTG | TCTGAACTG |
| scaffold_5 | 87962 | GATTAAGGG | GATTGAGGG | GATTGAGGG | GATTrAGGG | GATTrAGGG |
| scaffold_5 | 100211 | TCCTTGAAT | TCCTCGAAT | TCCTCGAAT | TCCTyGAAT | TCCTCGAAT |
| scaffold_5 | 350872 | GGCGTGCCC | GGCGyGCCC | GGCGyGCCC | GGCGTGCCC | GGCGyGCCC |
| scaffold_5 | 599922 | CGTCATTCA | CGTCrTTCA | CGTCrTTCA | CGTCATTCA | CGTCrTTCA |
| scaffold_5 | 851262 | TAATTCTCT | TAATCGTCT | TAATCGTCT | TAATysTCT | TAATCGTCT |
| scaffold_5 | 1099776 | ACATTGACA | ACATCGACA | ACATCGACA | ACATyGACA | ACATCGACA |
| scaffold_5 | 1352539 | TTGTGATCC | TTGTkrTCC | TTGTTGTCC | TTGTkrTCC | TTGTkrTCC |
| scaffold_5 | 1599904 | AACTTCCTT | AACTCCCTT | AACTCCCTT | AACTyCCTT | AACTCCCTT |
| scaffold_5 | 1851487 | TTCCGCTCC | TTCCsCTCC | TTCCGCTCC | TTCCGCTCC | TTCCsCTCC |
| scaffold_5 | 2100025 | CCCTTAGTC | CCCTyAGTC | CCCTCAGTC | CCCTyAGTC | CCCTyAGTC |
| scaffold_5 | 2278878 | GGTCGAAAA | GGTCrAAAA | GGTCAAAAA | GGTCGAAAA | GGTCrAAAA |
| scaffold_6 | 106480 | GCCCACTTG | GCCCrCTTG | GCCCGCTTG | GCCCrCTTG | syCCGCTTG |
| scaffold_6 | 350337 | CATTTGGTT | CATTyGGTT | CATTCGGTT | CATTyGGTT | CATTyGGTT |
| scaffold_6 | 600047 | GGAGCATTT | GGAGyATTT | GGAGyATTT | GGAGyATTT | GGAGCATTT |
| scaffold_6 | 849990 | AGTTCAGGA | AGTTyAGGA | AGTTyAGGA | AGTTyAGGA | AGTTCAGGA |
| scaffold_6 | 1098535 | CAAAGATTG | CAAArATTG | CAAArATTG | CAAArATTG | CAAArATTG |
| scaffold_6 | 1349453 | TGTCGGTAG | TGTCrrTAG | TGTCrrTAG | TGTCrrTAG | TGTCrrTAG |
| scaffold_6 | 1600000 | AAACCTGGA | AAACCTGGA | AAACCTGGA | AAACCTGGA | AAAymTGGA |
| scaffold_6 | 1764645 | AACCGGATT | AACCrGATT | AACCAGATT | AACCrGATT | AACCrGATT |
| scaffold_6 | 2000087 | GATTTTGCG | GATTTTGCG | GATTyTGCG | GATTyTGCG | GATTCTGCG |
| scaffold_6 | 2007502 | AATTGATAA | AATTrATAA | AATTAATAA | AATTGATAA | AATTAATAA |
| scaffold_7 | 348994 | CCGGAGTTT | CCGGmGTTT | CCGGwGTTT | CCGGmGTTT | CCGGmGTTT |
| scaffold_7 | 600111 | CAATTATTA | CAATyATTA | CAATTATTA | CAATyATTA | CAATyATTA |
| scaffold_7 | 850516 | TGACGCATA | TGACrCATA | TGACGCATA | TGACrCATA | TGACrCATA |
| scaffold_7 | 873221 | AATAGACCT | AATArACCT | AATAkACCT | AATArACCT | AATArACCT |
| scaffold_7 | 1100248 | TCACGGAAG | TCACrGAAG | TCrCrGAAG | TCACrGAAG | TCrCrGAAG |
| scaffold_7 | 1352529 | TAAATATAT | TAAATATAT | TAAAyATrT | TAAATATAT | TAAATATAT |
| scaffold_7 | 1605059 | GACAAGCAA | GACArGCAA | GACArGCAA | GACArGCAA | GACArGCAA |
| scaffold_7 | 1991524 | CAACCCACC | CAACyCACC | CAACyCACC | CAACCCACC | CAACyCACC |
| scaffold_8 | 350000 | ATTGACGCG | ATTGrCGCG | ATTGGCGCG | ATTGGCGCG | ATTGGCGCG |
| scaffold_8 | 606991 | GTGTATTCT | GTGTmTTCT | GTGTsTTCT | GTGTsTTCT | GTGTsTTCT |
| scaffold_8 | 610549 | GGAACTTGA | GGAAyTTGA | GGAATwyGA | GGAATwyGA | GGAATwyGA |
| scaffold_8 | 829832 | CTGTACAAC | CTGTrCAAC | CTGTrCAAC | CTGTrCAAC | CTGTrCAAC |
| scaffold_8 | 829846 | TTCGAGTGA | TTCGAGTGA | TTCGAGTGA | TTCGAGTGA | TTCGAGTGA |
| scaffold_8 | 830003 | AACTGGCAG | AACTGGCAG | AACTrGCAG | AACTrGCAG | AACTrGCAG |
| scaffold_8 | 830070 | ATTAGGATT | ATTAGGATT | ATTAGGATT | ATTAGGATT | ATTAGGATT |
| scaffold_8 | 830078 | TACTAGACG | TACTrGACG | TACTGGACG | TACTGGACG | TACTGGACG |
| scaffold_8 | 830105 | ATTTAGCAT | ATTTAGCAT | ATTTAGCAT | ATTTAGCAT | ATTTAGCAT |
| scaffold_8 | 830159 | AATTAGAAG | AATTAGAAG | AATTAGAAG | AATTAGAAG | AATTAGAAG |
| scaffold_8 | 830169 | GACGACTGG | GACGACTGG | GACGACTGG | GACGACTGG | GACGACTGG |
| scaffold_8 | 830215 | AGTGTATCT | AGTGyATCT | AGTGCATCT | AGTGCATCT | AGTGcATCT |
| scaffold_8 | 830250 | TCCAATGCA | TCCArTGCA | TCCAGTGCA | TCCAGTGCA | TCCAAGGCAG |
| scaffold_8 | 1100000 | CATACGATC | CATACGATC | CATACGATC | CATACGATC | CATACGATC |
| scaffold_8 | 1350240 | ACGGGTACT | ACGGrTACT | ACGGrTACT | ACGGrTACT | ACGGrTACT |
| scaffold_8 | 1354068 | AGAATGCCT | AGAAwGCCT | AGAAAGyyT | AGAAAGyyT | AGAAAGyyT |
| scaffold_8 | 1614036 | TTATCAGTA | TTATyAGTA | TTATyAGTA | TATyAGTA | TTATyAGTA |
| scaffold_8 | 1869238 | TGGAGGTTG | TGGAsGTTG | TGGAyGTTG | TGGAyGTTG | TGGAyGTTG |
| scaffold_9 | 00447 | CTATTTTCT | CTATkTTCT | CTATkTTCT | CTATsTTCT | CTATkTTCT |
| scaffold_9 | 350569 | AGAATATAC | AGAAAATAC | AGAATATAC | AGAArATAC | AGAATATAC |
| scaffold_9 | 599950 | TGGTATCCC | TsGTrTCCC | TGGTGTCCC | TGGTGTCCC | TGGTGTCCC |
| scaffold_9 | 611788 | TCTGTAATC | TTTGwrATC | TyTGTAATC | TTTGwrATC | TyTGTAATC |
| scaffold_9 | 721973 | TGTATACGT | TGTAGACGT | TGTAwACrT | TGTAGACGT | TGTAwACrT |
| scaffold_9 | 1010845 | GGGTGGTGA | GrGTGGTGA | GGGTGGTGA | GrGTrGTGA | GGGTGGTGA |
| scaffold_9 | 1250830 | TTGTGGGGA | TTGTAGGGA | TTGTGGGGA | TTGTwGGGA | TTGTGGGGA |
| scaffold_9 | 1499265 | AGTCAGACA | AGTCmGACA | AGTCsGACA | AGTCCGACA | AGTCsGACA |
| scaffold_9 | 1499300 | TATGACACC | TATGrCrCC | TATGACGCC | TATGACGCC | TATGACGCC |
| scaffold_9 | 1676755 | CTGCCGTTT | CTGCAGTTT | CTGCmGTTT | CTGCwGTTT | CTGCmGTTT |
| scaffold_9 | 1702348 | AGACGCATC | AGACrCATC | AGAmACATC | AGACACATC | AGAmACATC |
| scaffold_9 | 1702552 | CAAAGTCAT | CAAAGTCAT | CAAAGTCAT | CAAAGTCAT | CAAAGTCAT |
| scaffold_9 | 1702583 | ACTCAGCTG | ACTCAGCTG | ACTCAGCTG | ACTCAGCTG | ACTCAGCTG |
| scaffold_9 | 1702658 | TTGTCGTGG | TTGTTATGG | TTGTyrTGG | TTGTyATGG | TGTyrTTGT |
| scaffold_10 | 100470 | TCACCATCG | TCACyATCG | TCACyATCG | TCACCATCG | TCACCATCG |
| scaffold_10 | 350030 | GCGGCTCAA | GCGGyTCAA | GCGGTTCAA | GCGGyTCAA | GCGGTTCAA |
| scaffold_10 | 354531 | AATCAATCA | AATCmATCA | AATCmATCA | AATCmATCA | AATCmATCA |
| scaffold_10 | 633622 | TGGGCAAAG | TGGGsAAAG | TGGGrAAAG | TGGGsAAAG | TGGGrAAAG |
| scaffold_10 | 860249 | CCGCAAATT | CCGCrAATT | CCGCrAATT | CCGCAAATT | CCGCrAATT |
| scaffold_10 | 863401 | ATAAAATTT | ATAAAATTT | ATaAAwTTT | ATAAAATTT | ATaAAwTTT |
| scaffold_10 | 1107782 | CAACCCCAC | CAACCCCAC | CAACCCCAC | CAACCCCAC | CAACCCCAC |
| scaffold_10 | 1338596 | GTGCATCAT | GTGCwTCAT | GTGCyTCAT | GTGCmTCAT | GTGCyTCAT |
| scaffold_10 | 1477092 | AGATGCAAA | AGATsCAAA | ArATsCAAA | AGATGCAAA | ArATsCAAA |
| scaffold_10 | 1612161 | TCTTCGGAG | TCTTCGGAG | TCTTCGGAG | TCTTyGGAG | TCTTCGGAG |
| scaffold_10 | 1612569 | ATTATATTC | ATTATATTC | ATTATATTC | ATTATATTC | ATTATATTC |
| scaffold_10 | 1612630 | TGGCTCCTT | TGGCTCCTT | TGGCTCCTT | TGGCTCCTT | TGGCTCCTT |
| scaffold_10 | 1612671 | GGAATCGTC | GGAATCGTC | GGAATCGTC | GGAATCGTC | GGAATTCGT |
| scaffold_11 | 101855 | CCAGCCTGT | CCAGyCTGT | CCAGCCTGT | CCAGCCTGT | CCAGyCTGT |
| scaffold_11 | 173230 | AGCGGGCGA | AGCGGGCGA | AGCGrGCGA | AGCGsGCGA | AGCGGGCGA |
| scaffold_11 | 350000 | GTCAGCAAG | GTCAGCAAG | GTCAGCAAG | GTCAGCAAG | GTCAGCAAG |
| scaffold_11 | 378409 | TGATTGGGG | TGATkGGGG | TGATTGGGG | TGATwGGGG | TGATTGGGG |
| scaffold_11 | 600001 | TGGGCGCGC | TGGGmGCGC | TGGGmGCGC | TGGGmGCGC | TGGGmGCGC |
| scaffold_11 | 627221 | TCTTCGCCC | TCTTsGCCC | TCTTsGCCC | TCTTyGCCC | TCTTsGCCC |
| scaffold_11 | 929659 | GGAATATCA | GGAAkwTCA | GGAAkwTCA | GGAAkwTCA | GGAAkwTCA |
| scaffold_11 | 931877 | GACCTCACC | GACCkCACC | GACCkCACC | GACCkCACC | GACCkCACC |
| scaffold_11 | 1155850 | T-TGCCACG | TGTGyCACG | TATGCCACG | TATsCCACG | TgTGyCACG |
| scaffold_11 | 1240230 | ACAAGATTC | ACAArATTC | ACAArATTC | ACAAGATTC | ACAArATTC |
| scaffold_11 | 1250447 | GAGGCTACA | GAGGsTACA | GAGGsTACA | GAGGmTACA | GAGGsTACA |
| scaffold_12 | 109790 | GTCTGCACC | GTCTrCACC | GTCTrCACC | GTCTrCACC | GTCTrCACC |
| scaffold_12 | 272255 | CCGAGTGCT | CCGAmTGCT | CCGArTGCT | CCGArTGCT | CCGArTGCT |
| scaffold_12 | 281720 | CTTCCGGCG | CTTCTTCCG | CTTCyksCG | CTTCyksCG | CTTCyksCG |
| scaffold_12 | 281763 | TCTGCAGCC | TCTGyAGCC | TCTGyAGCC | TCTGyAGCC | TCTGyAGCC |
| scaffold_12 | 554582 | ACTCCGGTC | ACTCyGGTC | ACTCyGGTC | ACTCyGGTC | ACTCyGGTC |
| scaffold_12 | 770075 | GAACGTTCT | GAACATTCT | GAACrTTCT | GAACrTTCT | GAACrTTCT |
| scaffold_12 | 909536 | CTATGGAGG | CTATsGAGG | CTATsGAGG | CTATsGAGG | CTATsGAGG |
| scaffold_12 | 1000000 | CGAGGAGGA | CGAGrAGGA | CGAGrAGGA | CGAGrAGGA | CGAGrAGGA |
| scaffold_13 | 100697 | ACGTCTTTA | ACGTCTTTA | ACGTCTTTA | ACGTCTTTA | ACGTCTTTA |
| scaffold_13 | 119283 | ACGTTACTG | ACGyyACTG | ACGCsACTG | ACGTTACTG | ACGCsACTG |
| scaffold_13 | 363867 | ATCCACTGC | ATCCrCTGC | ATCCkCTGC | ATCCACTGC | ATCCkCTGC |
| scaffold_13 | 370521 | TTTGAGTCA | TTTGwGTCA | TTTGwGTCA | TTTGAGTCA | TTTGwGTCA |
| scaffold_13 | 604345 | CTTCAGCAT | CTTCAGCAT | CTTCAGCAT | CTTCAGCAT | CTTCAGCAT |
| scaffold_13 | 866136 | GTTGGTCAG | GTTGrTCAG | GTTGmTCAG | GTTGGTCAG | GTTGmTCAG |
| scaffold_14 | 113109 | AGGGAAATA | AGGGrAATA | AGGGrAATA | AGGGrAATA | AGGGrAATA |
| scaffold_14 | 372086 | CGATCCCTT | CGATyCCTT | CGATyCCTT | CGATyCyTT | CGATyCCTT |
| scaffold_14 | 603118 | GGCCCGCCT | GGCCmGCCT | GGCCmGCCT | GGCCsGCCT | GGCCmGCCT |
| scaffold_14 | 725687 | AGTTCGAAA | AGTTyGrAA | AGTTyGrAA | AGTTyGrAA | AGTTyGrAA |
| scaffold_14 | 808308 | AAGGTATGG | AAGswATGG | AAGsrATGG | AAGGTATGG | AAGsrATGG |
| scaffold_15 | 101381 | TAAACAGAT | TAAAyAGAT | TAAAwAGAT | TAAACAGAT | TAAAwAGAT |
| scaffold_15 | 150013 | GTGGCCCGT | GTGGmCCGT | GTGGmCCGT | GTGGCCCGT | GTGGmCCGT |
| scaffold_15 | 367204 | CGCGCCCTA | CGCGmCCTA | CGCGrCCTA | CGCGCCCTA | CGCGrCCTA |
| scaffold_16 | 106292 | AAGCTGGAA | AAGCmGGAA | AAGCmGGAA | AAGCTGGAA | AAGCmGGAA |
| scaffold_16 | 205778 | CAAGGTCTG | CAAGATCTG | CAAGATCTG | CAAGrTCTG | CAAGATCTG |
| scaffold_16 | 400000 | CCTCGGATT | CCTCGGATT | CCTCGGATT | CCTCGGATT | CCTCGGATT |
| scaffold_16 | 403998 | CAAAGTACG | CAAArTACG | CAAAGTACG | CAAArTACG | CAAArTACG |
| scaffold_17 | 134688 | CCCGCTTCA | CCCGyTTCA | CCCGyTTCA | CCCGyTTCA | CCCGyTTCA |
| scaffold_17 | 370858 | GACACAACG | GACAwAACG | GACAyAACG | GACATAACG | GACAyAACG |
| scaffold_17 | 449833 | ATCAGACAA | ATCAAACTA | ATCArACwA | ATCArACwA | ATCArACwA |
| scaffold_17 | 472545 | CCGTTCATG | CCGTyCrTG | CCGTCCGTG | CCGTCCGTG | CCGTCCGTG |
| scaffold_18 | 112940 | GCGGGTGGG | GCGGsTGGG | GCGGsTGGG | GCGGGTGGG | GCGGGTGGG |
| scaffold_18 | 126322 | CCTCTTCCG | CCTCwTCCG | CCTCwTCCG | CCTCkTCCG | CCTCkTCCG |
| scaffold_19 | 87323 | CCCAAGCAA | CCCAmGCAA | CCCAmGCAA | CCCAmGCAA | CCCAmGCAA |
| scaffold_19 | 98782 | AAAATTGTT | AAAAkTGTT | AAAAkTGTT | AAAAkTGTT | AAAAkTGTT |

A total of 199 SNP loci were utilized in each of Tables I and II. Note that there are two alleles (whether homo- or hetero-allelic) at each marker locus for the five heterokaryotic strains and a single allele per locus for the three homokaryotic cultures. The IUPAC nucleotide and ambiguity codes are used to represent the individual or composite 9-base DNA allelic marker sequences reported above, each of which represents an allele or a composite pair of alleles at a genomic marker locus in the indicated genomic position. The first two columns provide positional information of each marker locus from the haploid/homokaryotic reference genome H97 version 2.0, published by the U.S. Department of Energy Joint Genome Institute (Morin et al. 2012). H97 was originally obtained from a white cultivar strain. Genotypic differences are apparent among the strains. It is also clear that when the SNP patterns at each locus for B12998-s181 and P2s-203 are combined, the composite is a complete match with the B19414 heterokaryon. Also, marker scaffold 4, 753116 is not a classic 9-mer. This marker was included in the Tables simply because it was used in earlier patent applications, including PCT Application Publication No. WO2022023290A1.

Using the SNP (Single Nucleotide Polymorphism) data in Table I, it is possible to calculate an estimate of the genotypic differences between B19414 and the four other brown cultivar strains. Data in Table II shows that Tuscan-820 has 58/199 or 29.1% differences with B19414, Heirloom has 99/199 or 49.7% differences with B19414, Tuscan-860 has 51/199 or 25.6% differences with B19414, and Brawn closely follows Heirloom in having 109/199 or 54.8% differences with B19414. Thus, strain B19414 is substantially different from other available brown cultivar strains and provides both genetic diversification and commercially acceptable performance in terms of crop and product characteristics.

Using the complete set of SNP data that was captured for Scaffolds 1 through 19, it was also possible to estimate how similar the four brown strains were to the H97 reference genome, and to each other, using a process provided in the Lasergene software package. That estimation process has some limitations, in particular that all data are generated in comparison to a reference genome, in this case, that of H97. These tabulations provided by the software showed that B19414, Heirloom, Tuscan-820. Tuscan-860 and Brawn all have more than 380,000 SNP differences with H97. Furthermore, if we subtract the total SNPs reported for B19414 from the totals for each other strain, we can calculate the number of SNPs in Heirloom, Tuscan-820, Tuscan-860 and Brawn, which are different from those in B19414. Based on the absolute value of each difference, we arrived at an estimate of a minimum number of SNP differences distinguishing B19414 from each strain. These calculations showed that Tuscan-820 had 269,416 SNPs, Tuscan-860 had 157,704 SNPs, Heirloom had 272,938 SNPs, and Brawn had 128,212 SNPs different from the corresponding SNPs in B19414. These very large numbers demonstrate that B19414 has a unique and substantially different genotype compared to the available brown cultivars. There are some caveats to these calculations due to factors such as software limitations and DNA sequence read depth. Thus, the Applicant would like to stress that these numbers are approximations.

Tables III and IV report data from six genetic marker loci that are standardly reported, for same panel of strains utilized in Tables I and II. As in Tables I and II, there are two alleles at each marker locus for the five heterokaryotic strains and a single allele per locus for the three homokaryotic cultures. The data was prepared by the Applicant using targeted Polymerase Chain Reactions (PCR) to amplify genomic regions spanning the defined markers from each of the culture DNAs. PCR primers that bracket the defined marker regions, at locations indicated by the positional information provided below, were utilized to generate the data; methods of designing suitable primers are well known in the art. From the amplified PCR product, DNA was sequenced by a contractor, Eurofins (Louisville, Kentucky), using methods of their choice, and the genotypes were determined by direct inspection of these sequences in comparison to Sylvan's database of reference marker/allele sequences. In most cases the sequence was further confirmed by direct inspection of the corresponding whole genome sequence for that culture.

TABLE III
Genotypes of relevant cultures at six standard marker loci

| | | Line or Strain: | | | |
|---|---|---|---|---|---|
| | | H97 v | P2- | B12998 - | |
| Markers | Ref Pos | 2.0 | s203 | s181 | B19414 |
| p1n150-G3-2 | 868615 | 1T | 2 | 5 | 2/5 |
| (scaffold_1) | | | | | |
| ITS (scaffold_10) | 1612110 | I1 | I2 | I1 | I1/I2 |
| MFPC-ELF | 829770 | E1 | E4 | E3 | E3/E4 |
| (scaffold_8) | | | | | |
| AN (scaffold_9) | 1701712 | N1 | N3 | N1 | N1/N3 |
| AS (scaffold_4) | 752867 | SD | SA | SC | SA/SC |
| FF (scaffold_12) | 281674 | FF1 | FF1 | FF1 | FF1/FF1 |

TABLE IV
Genotypes of relevant comparison cultures at six standard marker loci

| | | Strain: | | | | | |
|---|---|---|---|---|---|---|---|
| | | H97 v | Tuscan- | | Tuscan- | Tuscan- | |
| Markers | Ref Pos | 2.0 | 820 | Heirloom | 860 | 860 | Brawn |
| p1n150-G3-2 (scaffold_1) | 868615 | 1T | 2/5 | 1T/5 | 2/5 | 2/5 | 1T/5 |
| ITS (scaffold_10) | 1612110 | I1 | I1/I5 | I1/I1 | I1/I2 | I1/I2 | I1 |
| MFPC-ELF (scaffold_8) | 829770 | E1 | E1/E3 | E3/E4 | E3/E4 | E3/E4 | E3/E4 |
| AN (scaffold_9) | 1701712 | N1 | N2/N3 | N2/N3 | N3/N4 | N3/N4 | N2/N3 |
| AS (scaffold_4) | 752867 | SD | SA/SD | SA/SD | SC/SD | SC/SD | SB/SD |
| FF (scaffold_12) | 281674 | FF1 | FF1/FF3 | FF1/FF3 | FF1/FF3 | FF1/FF3 | FF1/FF3 |

The data in Tables III and IV demonstrates that strain B19414 has a unique combination of alleles at this group of six standard marker loci. In particular, the genotypes at three of the six marker loci, FF, AN and AS, are not present in any of the other cultivars. Further analysis of these six markers are set forth below.

Description of the p1n150-G3-2 Marker:

The 5′ end of this marker segment begins at position 1 with the first “T” in the sequence TCCCAAGT, corresponding to H97 JGI V2.0 Scaffold 1 position 868615 (Morin et al. 2012) and extending in a reverse orientation (relative to the scaffold orientation) for ca. 600 nt in most alleles; an insertion in the DNA of allele 1T has produced a longer segment. At present, 9 alleles incorporating at least 30 polymorphic positions have been documented from diverse strains in Sylvan's culture collection.

Alleles present in the B19414 pedigree over three generations are alleles 2 and 5, characterized as follows (using the format: nucleotide base character @ alignment position):
Allele 1T: insertion of ABR transposon of 320 nt @206\hat{ }207, ‘A’ @321; ‘T’ @327; ‘C’ @374; ‘G’ @378; ‘G’ @422, ‘C’ @431; ‘G’ @472; etc.
Allele 2: no Abr1 insertion; ‘A’ @ 321; ‘C’ @ 327, ‘C’ @ 374; ‘C’ @ 378; ‘G’ @ 422; ‘T’ @ 431; ‘G’ @ 472; etc.
Allele 5: no Abr1 insertion; ‘G’ @321; ‘C’ @ 327, ‘C’ @ 374; ‘C’ @ 378; ‘G’ @ 422; ‘T’ @ 431; ‘G’ @ 472; etc.

Because of linkage to the MAT locus, which is obligately heteroallelic in fertile heterokaryons, genotypes of all known and expected heterokaryons at p1n150-G3-2 are also heteroallelic.

The genotype of the B19414 heterokaryon at the p1n150-G3-2 marker locus is ‘2/5’ (heteroallelic), designating the presence of alleles 2 and 5. Allele 2 was contributed by P2-s203, and allele 5 was transmitted from the B12998-s181. The ‘2/5’ genotype is also shared by Tuscan-820 and Tuscan-860. Heirloom and Brawn have alleles 1T and 5.

### Description of the ITS (=ITS 1+2 Region) Marker:

The ITS segment is part of the nuclear rDNA region which is located on chromosome 9 (Scaffold 10 in JGI H97 V2.0). The rDNA is a cassette that is tandemly repeated up to an estimated 100 times in the haploid genome of A. bisporus. Therefore, there is no single precise placement of this sequence in the assembled H97 genome, and in fact it is difficult or impossible to precisely assemble the sequence over all the tandem repeats. Three cassette copies were included on scaffold 10 of the H97 JGI V2.0 assembly, beginning at position 1612110; a partial copy is also assembled into scaffold 29 (Morin et al. 2012). The 5′ end of this marker segment begins at position 1 with the first “G” in the sequence GGAAGGAT and extending in a forward orientation (relative to the scaffold orientation) for ca. 703-704 nt in most alleles. At present, more than 9 alleles incorporating at least 11 polymorphic positions have been documented from diverse strains in Sylvan's culture collection.

Alleles present in the B19414 are I1 and I2. Characterized as follows (using the format: nucleotide base character @ alignment position, based on alignment of 9 alleles).
Allele I1: ‘C’ @ 52; ‘T’ @ 461; ‘T @ 522; ‘T’ @ 563; etc.
Allele I2: ‘T’ @ 52; ‘T’ @ 461; ‘T’ @ 522; ‘T’ @ 563; etc.
Allele I5: ‘G’ @ 52; ‘A’ @ 461: ‘C’ @ 522; ‘C’ @ 563; etc.

The genotype of the B19414 heterokaryon at the ITS marker locus is ‘I1/I2’ (heteroallelic), designating the presence of alleles I1 and I2. This pattern matches Tuscan-860 and is different from Heirloom, Tuscan-820 and Brawn.

### Description of the MFPC-1-ELF Marker:

The 5′ end of this marker segment begins at position 1 with the first “G” in the sequence GGGAGGGT, corresponding to H97 JGI V2.0 Scaffold 8 position 829770 (Morin et al. 2012) and extending in a forward orientation (relative to the scaffold orientation) for ca. 860 nt in most alleles. At present, at least 7 alleles incorporating at least 40 polymorphic positions have been documented from diverse strains in Sylvan's culture collection.

Alleles present in B19414 are E3 and E4
Allele E1: ‘A’ @63; ‘A’ @ 77; ‘A’ @ 232; ‘A’ @ 309; ‘T’ @ 334; ‘A’ @ 390; ‘A’ @ 400; ‘T’ @ 446, ‘A’ @ 481; etc.
Allele E3: ‘A’ @63; ‘A’ @ 77; ‘A’ @ 232; ‘G’ @ 309; ‘T’ @ 334; ‘A’ @ 390; ‘A’ @ 400; ‘C’ @ 446, ‘G’ @ 481; etc.
Allele E4: ‘G’ @63; ‘A’ @ 77; ‘A’ @ 232; ‘G’ @ 309; ‘T’ @ 334; ‘A’ @ 390; ‘A’ @ 400; ‘C’ @ 446; ‘G’ @ 481; etc.

The E3/E4 genotype matches Heirloom, Tuscan-860 and Brawn, however Tuscan-820 has a different genotype.

### Description of the AN Marker:

The 5′ end of this marker segment begins at position 1 with the first “G” in the sequence GGGTTTGT, corresponding to H97 JGI V2.0 Scaffold 9 position 1701712 (Morin et al. 2012) and extending in a forward orientation (relative to the scaffold orientation) for ca. 1660 (in the H97 genome) to 1700 nt (in alignment space) in known alleles; several insertions/deletions have created length polymorphisms which, in addition to point mutations of individual nucleotides, characterize the alleles. At present, 5 alleles incorporating more than 70 polymorphic positions have been documented from diverse strains in Sylvan's culture collection.

Alleles present are as follows (using the format: nucleotide base character @ alignment position, based on alignment of alleles N1 through N4):
Allele N1: ‘G’ @ 640; [deletion] @ 844-846; ‘C’ @ 954; T′ @882; ‘A’ @954, etc.
Allele N2: ‘A’ @ 640; [deletion] @ 844-846; ‘C’ @ 954; ‘T’ @882; ‘T’ @954, etc.
Allele N3: ‘A’ @ 640; [deletion] @ 844-846; ‘T’ @ 954; ‘C’ @882; ‘G’ @954, etc.
Allele N4: ‘A’ @ 640; [deletion] @ 844-846; ‘C’ @ 954; ‘C’ @882; ‘G’ @954, etc.

The ‘N1/N3’ genotype is unique to B19414; all other genotypes that were tested, including Heirloom, Tuscan-860, Tuscan-820 and Brawn, possessed a different allele combination at the AN locus.

### Description of the AS Marker:

The 5′ end of this marker segment begins at position 1 with the first “G” in the sequence GG(T/N)GTGAT, corresponding to H97 JGI V2.0 Scaffold 4 position 752867 (Morin et al. 2012) and extending in a forward orientation (relative to the scaffold orientation) for ca. 1620 (in the H97 genome) to 1693 nt (in alignment space) in known alleles; several insertions/deletions have created length polymorphisms which, in addition to point mutations of individual nucleotides, characterize the alleles. At present, 7 alleles incorporating more than 80 polymorphic positions have been documented from diverse strains in Sylvan's culture collection.

Alleles present in the B19414 immediate pedigree are alleles SA and SD. In the later cap diameter experiment below, Brawn was added as an extra commercial strain, and it possesses Allele SB, which is also described below. Markers are characterized in part as follows (using the format: nucleotide base character @ alignment position, based on alignment of alleles SA through SG):
Allele SA: ‘T’ @ 28; [deletion] @ 258-263; ‘G’ @ 275; [insertion]+‘TTTCCCAGC’+[insertion] @ 309-249; ‘C’ @ 404, etc.
Allele SB: ‘T’ @ 28; [deletion] @ 258-263; ‘T’ @ 275; [insertion]+‘TTTCCCAGC’+[insertion] @ 309-249; ‘C’ @ 440, etc.
Allele SC: ‘T’ @ 28; ‘GATATC’ @ 258-263; ‘G’ @ 275; [insertion]+‘TTTCTCAGC’+[insertion] @ 309-249; ‘C’ @ 404, etc.
Allele SD: ‘C’ @ 28; [deletion] @ 258-263; ‘T’ @ 275; [deletion] @ 309-249; ‘T’ @ 275; [deletion] @ 309-349, ‘T’ @ 404, etc.

The ‘SA/SC’ genotype is unique to B19414, and is not found in Heirloom, Tuscan-820. Tuscan-860, or Brawn.

### Description of the FF Marker:

The 5′ end of this marker segment begins at position 1 with the first “T” in the sequence TTCGGGTG, corresponding to H97 JGI V2.0 Scaffold 12 position 281999 (Morin et al. 2012) and extending in a forward orientation (relative to the scaffold orientation) for ca. 570 nt in most alleles. At present, 7 alleles incorporating at least 20 polymorphic positions have been documented from diverse strains in Sylvan's culture collection.

Both Alleles in the B19414 heterokaryons are the ‘FF1’ genotype.
Allele FF1: ‘CCG’ @ 48-50, ‘C’ @ 91, etc.
Allele FF3: ‘TTC’ @ 48-50; ‘T’ @ 91, etc.

B19414 was the only strain in the study to have the ‘FF1/FF1’ genotype.

A unique genotype, or genetic fingerprint, allows strain B19414 (and its EDVs and direct descendants) to be unambiguously identified. Agronomically, genetic diversity among cultivated strains is a desirable objective because it is well established that genetic monocultures among agricultural crop species can lead to disastrous failures due to particular disease, pest, or environmental pressures. Any otherwise desirable commercial strain with genetic novelty is therefore valuable. Strain B19414 meets those criteria.

TABLE V
B19414 versus Heirloom; yield over three breaks of production

Strain

| | B19414 | Heirloom | sd | p value |
|---|---|---|---|---|
| 1st Break | 3.6 | 3.98 | 0.25 | 3.65E−05**** |
| 2nd Break | 1.67 | 1.53 | 0.17 | 0.079 |
| 3rd Break | 1.1 | 0.8 | 0.18 | 1.34E−06**** |
| Total Yield | 6.37 | 6.31 | 0.21 | 0.56 |

Data is in lbs per sq ft;
10 replicates per strain
Statistical treatment was a pair-wise t-test.

For the data in Table V, and the tables below, significance is indicated by asterisks. For p=0.05 or less, *; for p=0.01 or less, **; for p=0.001 or less, ***, of p=0.0001 or less, ****.

Mushrooms were gathered in a European test farm, which uses modern Dutch technology for mushroom growing. In particular, Phase III compost incubation was utilized, supplement was added at casing, and the same parameters as those used for Heirloom, most notably the same temperature regime, CO2 management and timing of water application, were used. The crop was aired on day 4, and mushrooms for each of the three flushes were collected over three days of harvest.

As discussed above, there is a clear need for new brown strains of Agaricus bisporus which have yields that are equivalent to the current cultivars, such as the Heirloom strain. Data in Table V show that strain B19414 matches Heirloom for total yield. Strain B19414 had a significantly higher yield in third break. A tendency toward more balanced break yields, as shown by strain B19414, provides a corresponding improvement in product quality and value.

TABLE VI
Mushroom Cap Color
L*a*b Chroma meter values for four brown strains

| Strain | L value | sd | p value | a value | sd | p value |
|---|---|---|---|---|---|---|
| B19414 | 57.28 | 3.03 | / | 10.62 | 0.72 | / |
| HRLM | 60.35 | 2.42 | 0.001*** | 11.69 | 0.75 | 6.84E−05**** |
| Tuscan-860 | 57.45 | 2.13 | 0.84 | 11.55 | 0.95 | 0.0016** |
| Tuscan-820 | 68.52 | 4.27 | 2.14E−11**** | 10.6 | 0.98 | 0.94 |

| | b value | sd | p value |
|---|---|---|---|
| B19414 | −17.59 | 1.7 | / |
| HRLM | −20.65 | 1.75 | 3.13E−06**** |
| Tuscan-860 | −19.8 | 1.81 | 0.00043*** |
| Tuscan-820 | −20.02 | 1.26 | 1.39E−05**** |

Statistical treatment was a pair-wise t-test, comparison with B19414

Mushroom cap color was measured using a Minolta Chroma Meter CR-200. Sample size was twenty medium sized mushrooms of 30-40 mm in diameter. The L*a*b system was used, where “L” is a measure of brightness, with 100 being complete whiteness and 0 being complete blackness. For the other two measurements, “a” is a green/red axis, and “b” is a yellow/blue axis. For “a”, red values align on the positive side of the common axis, and green values align on negative values. In a similar fashion, on the “b” axis, yellow values are positive and blue values are negative.

B19414 had a similar ‘L’ value to Tuscan-860 and had a lower L (was significantly darker) than Heirloom and Tuscan-820.

Measurements of ‘a’ showed that B19414 was less red than Heirloom and Tuscan-860. There was no significant difference between B19414 and Tuscan-820.

The most informative data was collected at the ‘b’ parameter. Strain B19414 had a significantly lower b value than the other three strains.

TABLE VII

| Strain | Delta E value vs B19414 |
|---|---|
| HRLM | 3.35 |
| Tuscan-860 | 1.48 |
| Tuscan-820 | 9.66 |

In the Applicant's marketing research, darker brown mushrooms are often preferred by retailers and consumers. Note that even if two colors are statistically different based on chromameter measurements, they are not necessarily perceptibly different to the human eye. Perceptibility can be estimated by calculating the delta E value between the colors (Sharma, Wu & Dalal; Color Research and Application: 2004, pp 21-30). The delta E equation gives results in the range 0 to 100. Values between 2 and 10 are similar but perceptibly different at a glance, while values less than 2 require close observation to tell apart, and values greater than 10 are significantly different. Delta E can be calculated using one of several formulas, the most accurate being CIEDE2000, published by the CIE in 2001.

As shown in Table VII, mushrooms obtained from strain B19414 have a delta E value between 2 and 10 when compared with Heirloom and Tuscan-820, meaning their brown color is distinctive and can be differentiated at a glance. With lower L, a, and b values, B19414 mushrooms are noticeably darker than Heirloom and Tuscan-820.

TABLE VIII

| | Mushroom Dimension Measurements | | | | | | | | |
|---|---|---|---|---|---|---|---|---|---|
| | Cap Height | | | Cap Diameter | | | Stem Width | | |
| Strain | Avg | p value | sd | Avg | p value | sd | Avg | p value | sd |
| B19414 | 29.6 | — | 3.1 | 49.3 | — | 4.2 | 12.0 | — | 1.4 |
| HRLM | 28.2 | 0.10 | 2.0 | 47.5 | 0.1 | 3.1 | 15.4 | 3.4E−08**** | 1.6 |
| Tuscan-860 | 29.7 | 0.88 | 2.1 | 49.6 | 0.8 | 2.3 | 14.9 | 4.6E−08**** | 1.2 |
| Tuscan-820 | 28.2 | 0.08 | 1.4 | 48.6 | 0.5 | 3.0 | 15.9 | 1.6E−08**** | 1.9 |

| | Flesh Height | | |
|---|---|---|---|
| Strain | Avg | p value | sd |
| B19414 | 11.5 | — | 1.2 |
| HRLM | 15.1 | 1.22E−09**** | 1.5 |
| Tuscan-860 | 14.7 | 8.38−E09**** | 1.4 |
| Tuscan-820 | 15.8 | 1.82−E−13**** | 1.1 |

Statistical treatment was a pair-wise t-test

These are direct measurements made by the terms defined above. These measurements were used to define the proportion measurements in Table IX below.

TABLE IX

| | Mushroom Shape Ratios and Statistics | | | | | |
|---|---|---|---|---|---|---|
| | | | | Flesh | | |
| Strain | Cap Roundness | sd | p value | Thickness | sd | p value |
| B19414 | 0.57 | 0.11 | / | 0.39 | 0.05 | / |
| HRLM | 0.6 | 0.04 | 0.47 | 0.53 | 0.04 | 1.26E−11**** |
| Tuscan-860 | 0.59 | 0.04 | 0.35 | 0.49 | 0.04 | 5.62E−08**** |
| Tuscan-820 | 0.58 | 0.02 | 0.75 | 0.56 | 0.03 | 3.92E−14**** |

| | Stem Thickness | sd | p value |
|---|---|---|---|
| B19414 | 0.22 | 0.02 | / |
| HRLM | 0.3 | 0.02 | 1.56E−13**** |
| Tuscan-860 | 0.32 | 0.02 | 1.14E−11**** |
| Tuscan-820 | 0.33 | 0.03 | 4.61E−12**** |

Statistical treatment was a pair-wise t-test

Mushroom cap measurements in Table Vill were taken using Storm 3C301 digital calipers. Sample sizes of twenty medium sized mushrooms at commercial maturity (35-40 mm in diameter with closed veils) were harvested and measured to obtain values for cap diameter and cap height. The mushrooms were then cut in half vertically to measure flesh thickness and stem width. Ratios between these values were calculated to find Cap Roundness (cap height/cap diameter), Flesh Thickness (flesh thickness/cap diameter), and Stem Thickness (stem width/cap diameter). Data in Table IX show that B19414 has a similar cap roundness ratio to Tuscan-860, Heirloom/and Tuscan-820.

For the Flesh Thickness ratio, statistically significant differences were observed when comparing the flesh thickness of strain B19414 to the flesh thickness of Heirloom, Tuscan-820 and Tuscan-860. B19414 had a Flesh Thickness ratio of 0.39, which was significantly lower than the other three strains in Table IX. In other words, B19414 had proportionally thinner cap flesh. This is a key difference, because it is indicative that as the mushroom matures it will flatten out rather than retain a round shape. As discussed above, there are lucrative markets in North America and Europe for brown strains that make good breakfast flats or portobellos.

Additionally, strain B19414 also has a lower Stem Thickness ratio than Tuscan-860, Heirloom, and Tuscan-820. Mushrooms of the same cap diameter have narrower stems, meaning more of the harvested mushroom is comprised of cap tissue, and less might be discarded as stem during kitchen preparation.

TABLE X
Closed Cap Mushroom Shelf Life

| | | Measurements and Statistics | | | | | |
|---|---|---|---|---|---|---|---|
| | Strain | Avg D4 | p value | Avg D5 | p value | Avg D6 | p value |
| 1st Flush | B19414 | 94.90% | 0.58 | 94.10% | 0.51 | 93.30% | 0.24 |
| Test 1 | HRLM | 94.80% | | 94.30% | | 93.50% | |
| 1st Flush | B19414 | 84.60% | 0.08 | 80.50% | 0.09 | 77.30% | 0.05* |
| Test 2 | HRLM | 82.50% | | 77.90% | | 73.80% | |

| | | Avg D7 | p value | Avg D8 | p value |
|---|---|---|---|---|---|
| 1st Flush | B19414 | 92.40% | 0.03* | 91.60% | 0.03* |
| Test 1 | HRLM | 92.90% | | 92.30% | |
| 1st Flush | B19414 | 73.90% | 0.03* | 70.80% | 0.03* |
| Test 2 | HRLM | 70% | | 66.70% | |

Statistical treatment was a pair-wise t-test.

Weight loss at 4° C. over a period of eight days was compared between strain B19414 and Heirloom. Close cap mushrooms were used at commercial maturity (around 40 mm in diameter with closed veils) during first break only.

The containers were Styrofoam wrapped with plastic film, each of which contained 300 g of mushrooms. These studies replicated what occurs in a supermarket setting, where mushrooms can be in a refrigerated display case for a period of up to ten days.

Data in Table X clearly shows that strain B19414 lost less moisture than the Heirloom control, in days 6, 7 and 8 in Test 1, and in days 7 and 8 in Test 2. This information is important because loss of water causes desiccation of the mushrooms, condensation within the over-wrapped mushroom containers, and reduced product weight at sale. These data further demonstrate one way in which strain B19414 produces closed button mushrooms having unexpected superior quality.

In confrontation cropping tests between strain B19414 and other commercial brown strains, typical heterokaryon incompatibility behaviors, including delayed timing and reduced yield of first break, were observed.

In Table XI below, a study was conducted to measure differences in maximum cap diameter between five different strains of brown mushroom grown as portobellos. Bulk Phase II compost was purchased from a commercial supplier (Kennett Square, Pennsylvania), spawn was mixed in and a 13-day spawn run was completed. A total of four 4 ft×4 ft trays were spawned for each strain. The casing layer (peat buffered with Ca CO3, PH ˜7.5, ˜75% H2O), mixed with the relevant casing spawn was applied to each tray at the conclusion of spawn run.

To grow portobello mushrooms, a grower will make some subtle changes to regular growing conditions. In the example of the data shown in Table XI, the growing room was flushed on day 6 by dropping the CO2 from 4000 ppm to 1400 ppm over a 24-hour period. In addition to the CO2 change, air temperature was dropped from 22° C. to 16.5° C., and the relative humidity dropped from 95% to 82%. Water was also applied to the casing layer of all strains in the day of flush, and water was applied as necessary as the crop developed. Taken together, these growing conditions are described as a soft flush. Small mushrooms were ready to harvest on day 15 post-case.

Trays were managed to produce portobello mushrooms. Mushroom density on the trays was reduced by trained harvesters to allow the mushrooms space to achieve maximum size in all trays. The key metric was to harvest large mushrooms on the day that the veil broke away from the cap margin. This is when a commercial grower will typically harvest portobello mushrooms. If the mushrooms are left on the beds past the point of veil rupture, the cap margin will begin to curl upwards, and spores will start to accumulate on the surface of the beds. Growers do not want to harvest mushrooms with an upturned cap margin and favor a cap margin where the underside is “rolled under”. Additionally, open mushrooms present a risk for the spread of mushroom viruses, and spores on mushroom beds are known to depress yield.

For portobello mushrooms, mushroom growing is a balancing act between manipulating the number of pins on the surface of the beds to encourage the growth of larger mushrooms which retain an intact or largely intact veil at harvest. Therefore, in the data described below in Table XI, the key metric was to harvest large mushrooms on the day that the veil began to break away from the cap margin. All of the mushrooms harvested in Table XI below had intact veils on the day of harvest. However, the veil was beginning to stretch and they could not have been left in the mushroom house for an additional day.

TABLE XI
Cap Diameter of Large Mushrooms in six brown cultivars

| Strain | N | Mean | sd | p value | Class |
|---|---|---|---|---|---|
| B19414 | 27 | 105.45 | 8.68 | — | a |
| Brawn | 37 | 97.75 | 8.71 | 9.54E−04*** | b |
| Tuscan-820 | 31 | 97.10 | 7.12 | 1.89E−04*** | b |
| HRLM | 30 | 93.30 | 6.24 | 3.06E−08**** | c |
| Tuscan-860 | 28 | 86.00 | 4.64 | 1.51E−13**** | d |

For the data in Table XI, five strains were used in the cap diameter study. In the Table, N is the number of mushrooms harvested on the day of data capture. The Mean is the mean (average) cap diameter, in millimeters, for all of the N number of mushrooms harvested, while sd is the standard deviation (a measure of the variation in each data set). For the p values, significance is indicated by asterisks. For p=0.001 or less, ***, of p=0.0001 or less, ****. The column labeled as Class in Table XI shows the results of a full t-test comparison between all five strains. Four statistically distinct classes were observed. It is noted that Brawn and Tuscan-820 were not significantly different from one another, and thus, were both set forth in class b.

In Table XI, the statistical treatment that calculated the p values was a pairwise t-test, with B19414 being compared with each of the other five strains separately. These data show a clear, highly significant difference between B19414 and the other five strains. We therefore conclude that B19414 can be harvested at a larger size than all the other brown commercial strains of Agaricus bisporus.

In summary, in Table XI, it can clearly be seen that strain B19414 made the largest portobello mushrooms. Large portobellos are a premium grade of mushroom that will typically fetch a higher price. Hence there are clear market advantages for B19414 over other cultivars.

Although the invention has been described in terms of particular embodiments in this application, one of ordinary skill in the art, in light of the teachings herein, can generate additional embodiments and modifications without departing from the spirit of, or exceeding the scope of, the claimed invention. Accordingly, it is understood that the descriptions herein are proffered only to facilitate comprehension of the invention and should not be construed to limit the scope thereof.

1. A mushroom culture of Agaricus bisporus designated as strain B19414, a representative culture of the strain having been deposited under NRRL Accession No. 68095.

2. A culture of Agaricus bisporus having a genotypic fingerprint which has alleles at marker loci ITS, p1n150-G3-2, MFPC-1-ELF, AN, AS, and FF, wherein all of the alleles at marker loci ITS, p1n150-G3-2, MFPC-1-ELF, AN, AS, and FF of said fingerprint are present in the genotypic fingerprint of strain B19414, wherein a culture of strain B19414 has been deposited under the NRRL Accession Number 68095.

3. A culture of Agaricus bisporus derived directly from an initial culture of strain B19414, wherein a culture of the strain has been deposited under NRRL Accession No. 68095, such that all of its genome or genotype is present in the genome or genotype of the initial culture of strain B19414.

4. A culture of Agaricus bisporus derived from an initial culture, wherein said initial culture is the culture of claim 1, such that at least 75% of the genome or genotype of the derived culture is present in the genome or genotype of the initial culture of strain B19414, wherein a culture of the strain has been deposited under NRRL Accession No. 68095.

5. The culture of claim 1, wherein said culture produces a crop of first break portobello mushrooms having brown caps, and having a cap diameter wider than the cap diameter of a cultivar strain selected from the group consisting Heirloom, Tuscan-820, Tuscan-860 and Brawn, at the same stage of maturity.

6. The culture of claim 1, wherein said culture produces a crop of mushrooms having brown caps and having a Flesh Thickness ratio less than 0.49.

7. A part of the mushroom culture of claim 1, selected from the group consisting of hyphae, spores, cells, and parts of cells, selected from nuclei and protoplasts, wherein, when the part are cells, the cells are formed upon the basidial cell of a mushroom of the culture of Agaricus bisporus designated as strain B19414, a representative culture of the strain having been deposited under NRRL Accession No. 68095, and wherein, when the part are spores, the spores are selected from dormant and germinated spores, and wherein the dormant and germinated spores include heterokaryons and homokaryons.

8. A product comprising the mushroom culture of claim 1, the product selected from the group consisting of mycelium, spawn, inoculum, casing inoculum, fresh mushrooms, processed mushrooms, mushroom pieces, and colonized substrates including grain, compost, and friable particulate matter.

9. The mushroom culture of claim 2, wherein said culture has a genotypic fingerprint which has allelic characters at marker loci described in a column labeled B19414 of Table I, wherein all of the allelic characters of said fingerprint are present in the genotypic fingerprint of strain B19414, wherein a culture of strain B19414 has been deposited under the NRRL Accession Number 68095.

10. A culture which is a line obtained from the part of the mushroom culture of claim 7.

11. A line incorporating a part of the mushroom culture of claim 7.

12. Heterokaryons and homokaryons obtained from the mushroom culture of claim 1.

13. Spores obtained from the mushroom culture of claim 1.

14. Mushrooms obtained from the mushroom culture of claim 1.

15. A method of producing a mushroom culture of Agaricus bisporus comprising mating a homokaryotic line designated B12998-s181, a culture of which has been deposited under NRRL Accession No. 68094, with a homokaryotic line designated P2-s203, a culture of which has been deposited under NRRL Accession No. 68093.

16. A cell of the mushroom culture of claim 1.

17. A culture comprising the cell of claim 16.

18. (canceled)

19. (canceled)

20. (canceled)

21. (canceled)
